SEQUENCE LISTING (1 sequence)

SEQ ID NO: 1 - protein, 154 aa, Equus caballus
MGLSDGEWQQVLNVWGKVEADIAGHGQEVLIRLFTGHPETLEKFDKFKHLKTEAEMKASEDLKKHGTVVLTALGGILKKKGHHEAELKPLAQSHATKHKIPIKYLEFISDAIIHVLHSKHPGDFGADAQGAMTKALELFRNDIAAKYKELGFQG

(19) United States
(12) Patent Application Publication
(10) Pub. No.: US 2025/0162912 A1
(43) Pub. Date: May 22, 2025

(54) METAL CHELATORS

(71) Applicant: Wayne State University, Detroit, MI (US)

(72) Inventor: Jeremy KODANKO, Grosse Pointe, MI (US)

(21) Appl. No.: 18/515,982

(22) Filed: Nov. 21, 2023

(51) Int. Cl. C02F 1/54; C02F 101/20; C07K 1/107

(52) U.S. Cl. CPC C02F 1/54; C07K 1/1077; C02F 2101/20; C02F 2303/16

(57) ABSTRACT

A chelator having formula I or a salt thereof removing metal ions from a solution is provided:

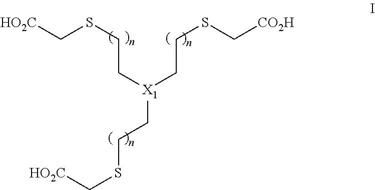

wherein X₁ is N or P; and n is 1 or 2. Protein-chelator systems for removing metal ions from a solution are also provided.

### STATEMENT REGARDING FEDERALLY SPONSORED RESEARCH OR DEVELOPMENT

The invention was made with Government support under Contract No. W912HZ-21-2-0048 awarded by the U.S. Army Engineer Research and Development Center. The Government has certain rights to the invention.

### SEQUENCE LISTING

The XML file WSU0250PUS.xml of size 1,761 bytes created Nov. 20, 2023 filed herewith, is hereby incorporated by reference.

### TECHNICAL FIELD

In at least one aspect, the present invention is related to compounds for chelating transition metals and lanthanide metals.

### BACKGROUND

Transition metals have important applications in the fields of catalysis, electronics, electric vehicles, and ceramics and are vital for maintaining a robust economy.¹⁻⁷ The main source of transition metals is mining them from their ores. However, mining is a destructive process responsible for environmental hazards including loss of biodiversity and production of toxic gases.^{8,9} Owing to these environmental hazards, alternative sources for transition metals are desperately needed. Fly ash produced from combustion of coal is a rich source of lanthanides and is also known to contain large amounts of transition metals like Ni, Cu, Zn, Fe, Pb, and V.¹⁰⁻¹³ Acid leeching is one of the most common techniques used to recover lanthanides from coal fly ash with new strategies being developed.¹⁴⁻¹⁶ However, little work has been carried out to recover other valuable transition metals from these leachates. Moreover, after recovering the lanthanides from coal fly ash, the remaining transition metals pose additional disposal challenges and an opportunity to recover other valuable metals.

In this regard, Ni has the unique ability to bind to both hard and soft donor ligands, which helps it form various different types of geometries, including square planar (four coordinate), square pyramidal (five coordinate), and octahedral (six coordinate).¹⁷ Schiff base-type ligands containing N and O donor atoms are known to selectively bind to Ni in aqueous solution and hence are used as sensors for Ni.^{18,19} A BODIPY-based fluorescent Ni(II) sensor (NS-1) containing N, S, and O donor atoms is able to bind to and detect Ni(II) selectively in living cells (FIG. 1).²⁰ NS-1 also showed high affinity toward Ni(II) with a K_{d} value of 196 μM in aqueous solution. Recently, the related Ni(II) chelator Nicastro was reported (FIG. 1B).²¹ The salient feature of NiCast is its photochemical properties. Upon irradiating Ni(II) bound NiCast, [Ni(NiCast)] with light, Ni(II) is released, and the nitrobenzhydrol group of NiCast is converted to a benzaldehyde group. Although NiCast is selective for Ni(II), its affinity in aqueous medium (K_{d} value of 560 μM) is less than that of NS-1. Importantly, neither NS-1 nor NiCast showed selectivity toward Ni(II) over Cu(II).

Accordingly, there is a need for improved chelators that address the challenges of the prior art.

### SUMMARY

In at least one aspect, a chelator for removing transition metal or lanthanide metal ions from a solution is provided. The chelator includes a compound having formula I:

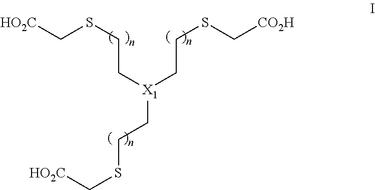

wherein:
X₁ is N or P; and
n is 1 or 2.

In another aspect, a protein-chelator system for removing transition metal or lanthanide metal ions from a solution is provided is provided. The protein-chelator system includes a chelating moiety having formula I or II or salts thereof conjugated to a protein having at least lysine residue. Advantageously, the chelating moiety is conjugated through an amide bond formed from a nitrogen atom in the at least one lysine residue and a carboxylate moiety in the chelating moiety having formula I or III:

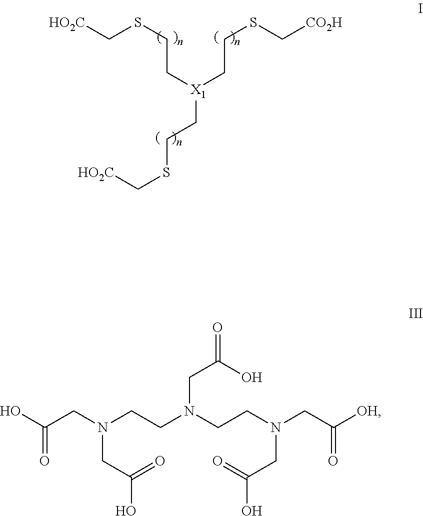

wherein:
X₁ is N or P; and
n is 1 or 2.

In another aspect, methods using the compounds having formulas I, II, and III for removing transition metal or lanthanide metal ions from a solution is provided

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1A. Structures of nickel chelators containing N, S, and O donor atoms: NS-1, NiCast, and the trisodium salt SG-20.

FIG. 1B. Scheme 1: synthesis of selective Ni(II) chelator SG-20.

FIG. 2. Determination of speciation of SG-20 (termed L) on increasing pH of the solution with the help of PHREEQC modeling. Binding studies of SG-20 with different metal ions.

FIGS. 3A and 3B. (A) Hill plot of absorbance change at λₘₐₓ=260 nm when SG-20 (25 μM) is titrated against Ni(II) in HEPES buffer (pH=7.1). (B) Linearity in the initial points of the Hill plot of absorbance change at λ=260 nm when SG-20 (25 μM) is titrated against Ni(II) in HEPES buffer (pH=7.1).

FIGS. 4A, 4B, 4C, and 4D. Hill plot of absorbance change at different wavelengths when various concentrations of SG-20 are titrated against (A) Ni(II) (5 μM, λ=260 nm) in HEPES buffer (pH=7.1), (B) Ni(II) (25 μM, λ=260 nm) in acetate buffer (pH=5.6), (C) Cu(II) (25 μM, λ=350 nm) in HEPES buffer (pH=7.1), and (D) Fe(II) (25 μM, λ=280 nm) in acetate buffer (pH=5.6).

FIGS. 5A, 5B, 5C, and 5D. Changes in the electronic absorption spectra when various concentrations of SG-20 are titrated against (A) Ni(II) (5 μM) in HEPES buffer (pH=7.1), (B) Ni(II) (25 μM) in acetate buffer (pH=5.6), (C) Fe(II) (25 M) in acetate buffer (pH=5.6), and (D) Cu(II) (25 μM) HEPES buffer (pH=7.1).

FIG. 6. XPS survey spectrum recorded for SG-20.

FIG. 7. XPS survey spectrum recorded for Ni-SG-20.

FIG. 8. ORTEP image of NiClust-SG-20 with displacement ellipsoids at 50% (red=O, blue=N, yellow=S, gray=C, purple=Na, and green=Ni). H atoms and the solvated Na+ were removed for clarity.

FIGS. 9A, 9B, and 9C. (A) Structure of Ni₄(μ₃—OH)₂(H₂O)₆(NTP)₂], (B) structure of Ni-1′-Ac₂, and (C) partial structure of NiClust-SG-20, showing the binding of one outer rim octahedral Ni center, with a third arm of SG-20 bridging other Ni(II) ions within a cluster.

FIG. 10. Structure of SG-20 and depiction of SG-20 binding to Ni(II) characterized by X-ray crystallographic analysis of a Ni(II) cluster.²⁶

FIGS. 11A and 11B. (A) MALDI-MS of Mb-SG20. (B) SDS-PAGE gel electrophoresis of Mb and Mb-SG-20. The Mb standard and Mb-SG20 sample were mixed with 4× loading buffer with beta-mercaptoethanol (BME) and about 5 μg of protein was loaded on to a 4-20% gradient gel. The samples were run in Tris/glycine buffer at 200 mV for 40 min. Afterward, the gel was fixed, stained in Coomassie Blue G250, and de-stained. Lane 1=Mb-SG20, lane 2=Mb, and lane 3=standard protein ladder.

FIGS. 12A and 12B. (A) Structure of Mb showing the surface Lys residues marked in green and Lys residues conjugated to SG-20 marked in red. The heme group is removed for clarity. (B) Amino acid sequence of Mb with all the Lys residues marked in green, and the Lys residues identified to be conjugated to SG-20 marked in red, with the structure of the proposed amide linkage between SG-20 and a Lys residue shown below.

FIGS. 13A, 13B, 13C, and 13D. (A) Ni concentrations (in ppb) observed when size exclusion filtration is performed with different concentrations of Ni(II) (20-80 μM) and Mb-SG-20 (designated as P) (5-40 μM) in PBS buffer (pH=7.1). P values are with respect to the concentration of Ni(II) (20-80 μM) in the absence of Mb-SG-20. ***P<0.01, **P<0.05, and *P<0.1. (B) Hill plot of change in the concentration of Ni(II) depleted using different concentrations of Mb-SG-20 (0-40 μM) in a solution with fixed Ni(II) concentration (80 μM); fit is based on the overall concentration of Mb-SG-20. (C) Cu concentrations (in ppb) observed when size exclusion filtration is performed with different concentrations of Cu(II) (20-80 μM) and Mb-SG-20 (designated as P) (5-40 μM) in PBS (pH=7.1). P values are with respect to the concentration of Cu(II) (20-80 μM) in the absence of Mb-SG20. ***P<0.01, **P<0.05, and *P<0.1. (D) Hill plot of change in the concentration of Cu(II) depleted using different concentrations of Mb-SG-20 (0-40 μM) in a solution with fixed Cu(II) concentration (80 μM); fit is based on the overall concentration of Mb-SG-20. Data are the average of three independent measurements plus or minus the standard deviation of the mean.

FIGS. 14A and 14B. (A) Ni concentrations (in ppb) observed when size exclusion filtration is performed with fixed concentrations of Ni(II) (40 μM) and Mb-SG-20 (designated as P) (10 μM) followed by treatment with aqueous EDTA (pH=7.1) and subsequent treatment with Ni(II). (B) Cu concentrations (in ppb) observed when size exclusion filtration is performed with fixed concentrations of Cu(II) (60 μM) and Mb-SG-20 (designated as P) (20 μM) followed by treatment with aqueous EDTA (pH=7.1) and subsequent treatment with Cu(II). Data are the average of three independent measurements plus or minus the standard deviation of the mean.

FIGS. 15A, 15B, 15C, and 15D. Concentrations of different metal ions in CFA leachate at pH=5.5 before and after treatment with Mb-SG-20: (A) 20, (B) 10, and (C,D) 5 μM. The data show the depletion of Ni and Cu ions after treatment of the CFA leachate with Mb-SG20. Data are the average of three independent measurements plus or minus the standard deviation of the mean.

FIG. 16. Structure of chelators containing N, S, and O donor atoms: NS-1 and NiCast.

FIGS. 17A, 17B, and 17C. A) Cartoon representation of europium capture process with the soluble protein capture agent Mb-DTPA; B) MALDI data for Mb-DTPA (upper) and Mb (lower); C) Efficiency of Eu3+ capture with Mb-DTPA over three cycles. Eu³⁺ concentrations, determined by emission assay, when size exclusion filtration is performed with Eu3+ (3 mM) and Mb-DTPA (designated as P) (0.3 mM) in MOPS buffer (pH=7.6), exchange with phosphate buffer (pH 7.4) leads to precipitation of Eu₂ (PO4)3 (inset); process carried out over three cycles with fresh Eu3+ solutions shows efficiency of recycling.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: all R groups (e.g. Rᵢ where i is an integer) include hydrogen, alkyl, lower alkyl, C₁₋₆ alkyl, C₆₋₁₀ aryl, C₆₋₁₀ heteroaryl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups M is a metal atom (e.g., Na, K, Li, etc.) and L− is a counter anion (e.g., Cl−, Br−, tosylate, etc.); single letters (e.g., “n” or “o”) are 1, 2, 3, 4, or 5; in the compounds disclosed herein including compounds described by formula or by name, a CH bond can be substituted with alkyl, lower alkyl, C₁₋₆ alkyl, C₆₋₁₀ aryl, C₆₋₁₀ heteroaryl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups M is a metal atom (e.g., Na, K, Li, etc.); percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

The term “alkyl” refers to C₁₋₂₀ inclusive, linear (i.e., “straight-chain”), branched, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The phrase “composed of” means “including” or “comprising.” Typically, this phrase is used to denote that an object is formed from a material.

The term “substantially identical” means nucleotide sequence with similarity to the nucleotide sequence of myoglobin (SEQ ID NO: 1). The term “substantially identical” can also be used to describe similarity of polypeptide sequences such as that to SEQ ID NO: 1 for myoglobin. For example, polypeptide sequences that are at least 70%, 75%, 80%, 85%, 90%, 92%, 95%, 96%, 98% or 99% identical to SEQ ID NO: 1 are also contemplated below.

To determine the “percent identity” (i.e., percent sequence identity) of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a refinement, the sequences are aligned for maximum correspondence over a specified comparison window, as measured by sequence comparison algorithms or by visual inspection. In a refinement, the length of a first sequence aligned for comparison purposes is at least 80% of the length of a second sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5. In this regard, the following oligonucleotide alignment algorithms may be used: BLAST (GenBank URL: www.ncbi.nlm.nih.gov/cgi-bin/BLAST/, using default parameters: Program: BLASTN; Database: nr; Expect 10; filter: default; Alignment: pairwise; Query genetic Codes: Standard(1)), BLAST2 (EMBL URL: http://www.embl-heidelberg.de/Services/index.html using default parameters: Matrix BLOSμM62; Filter: default, echofilter: on, Expect: 10, cutoff: default; Strand: both; Descriptions: 50, Alignments: 50), or FASTA, search, using default parameters. When sequences differ in conservative substitutions, the percent identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. In a variation, the polypeptides of SEQ ID NO: 1 with 1 to 7 conservative substitutions are also contemplated below.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

Throughout this application, where publications are referenced, the disclosures of these publications in their entirety are hereby incorporated by reference into this application to more fully describe the state of the art to which this invention pertains.

### Abbreviations

“CFA” means coal fly ash.

“DTPA” means diethylenetriamine pentaacetate.

“EDTA” means ethylenediaminetetraacetic acid.

“HEPES” means (4-(2-hydroxyethyl)-1-piperazineethanesulfonic acid.

“MALDI” means matrix-assisted laser desorption/ionization.

“MOPS buffer” means 3-(N-morpholino) propanesulfonic acid buffer.

“MWCO” means molecular weight cut-off.

“NTP” means nitrilotripropionic.

The term “conjugated to” means that a first chemical moiety is directed or indirectly covalently bonded to a second chemical moiety.

The term “transition metal” means an element whose atom has a partially filled d sub-shell, or which can give rise to cations with an incomplete d sub-shell. Examples of transition metals includes scandium, titanium, vanadium, chromium, manganese, iron, cobalt, nickel, copper, yttrium, zirconium, niobium, molybdenum, technetium, ruthenium, rhodium, palladium, silver, hafnium, tantalum, tungsten, rhenium, osmium, iridium, platinum, and gold.

The term “lanthanide” or “lanthanoid metal” means an element with atomic numbers 57-71. The lanthanides metals includes lanthanum, cerium, praseodymium, samarium, europium, gadolimium neodymium, promethium, terbium, dysprosium, holmium, erbium, thulium, ytterbium, or lutetium.

In an embodiment, a chelator for removing transition metal or lanthanide metal ions is provided. The chelator includes a compound having formula I or a salt thereof:

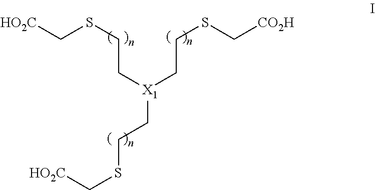

wherein:
X₁ is N or P; and
n is 1 or 2. In a refinement, X₁ is N. In a further refinement, n is 1. In a refinement, the salt is a sodium or potassium salt.

In another aspect, the chelator has formula II or a salt thereof for removing transition metal or lanthanide metal ions comprising:

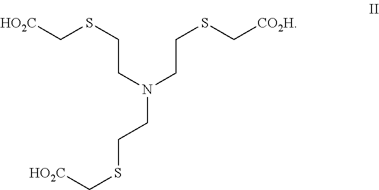 [SMILES: O=C(O)CSCCN(CCSCC(=O)O)CCSCC(=O)O]

In another embodiment, a protein-chelator system for removing transition metal or lanthanide metal ions from a solution is provided. is provided. The protein-chelator system includes a chelating moiety having formula I or II or salts thereof conjugated to a protein having at least lysine residue. Advantageously, the chelating moiety is conjugated through an amide bond formed from a nitrogen atom in the at least one lysine residue and a carboxylate moiety in the chelating moiety having formula I or III (DTPA):

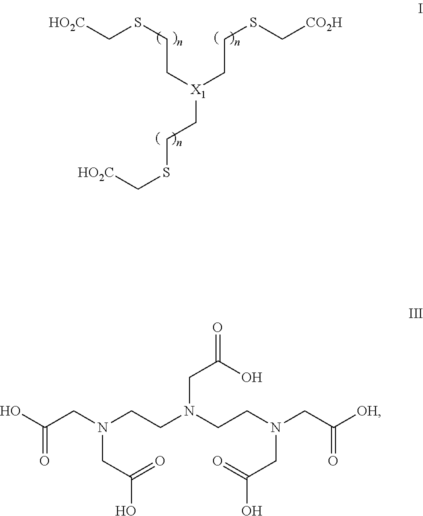

wherein:
X₁ is N or P; and
n is 1 or 2. In a refinement, X₁ is N. In a further refinement, n is 1. In a refinement, the salt is a sodium or potassium salt.

In another aspect, the protein includes a plurality of lysine residues. In a refinement, the protein includes at least 3 lysine residues. In another refinement, the protein includes at least 5 lysine residues. In some refinements, the protein includes at least 1, 2, 3, 4, 5, 6, 7, 8, 9 or 10 lysine residues. In further refinements, the protein includes at most 30, 25, 23, 20, 18, 15, 13, or 10 lysine residues.

In another aspect, the plurality of lysine residues includes at least one lysine residue positioned at the surface of the protein. In some refinements, the plurality of lysine residues includes at least 1, 2, 3, 4, or 5 lysine residues positioned at the surface of the protein. In a refinement, In further refinements, the protein includes at most 15, 13, 10, 8, or 5 lysine residues positioned at the surface of the protein. Although virtually any protein having lysine residues can be used, useful examples include myoglobin and albumin. (see, Schmiedl U, Ogan M, Paajanen H, Marotti M, Crooks L E, Brito A C, Brasch R C. Albumin labeled with Gd-DTPA as an intravascular, blood pool-enhancing agent for MR imaging: biodistribution and imaging studies. Radiology. 1987 January; 162(1 Pt 1):205-10. doi: 10.1148/radiology.162.1.3786763. PMID: 3786763; the entire disclosure of which is hereby incorporated by reference.)

In another embodiment, a method for removing metal ions from a solution is provided. The method includes a step of providing a target solution including transition metal or lanthanide metal ions. The target solution is contacted with a chelator having formula I or a salt thereof:

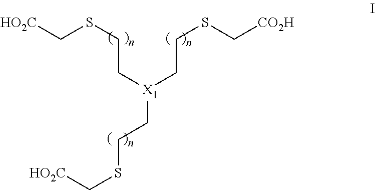

wherein:
X₁ is N or P; and
n is 1 or 2.
   The method also includes a step of collecting a precipitate that includes the transition metal ions and/or lanthanide metal ions. Examples of transition metal ions include Ni ions (e.g., Ni(II)) and/or Cu ions (e.g., Cu(II)). Examples of lanthanide metal ions include europium ions (e.g., Eu(II)). In a refinement, the salt is a sodium or potassium salt.

In another aspect, the precipitate with a release agent to recover the transition metal ions and/or lanthanide metal ions in solution. In a refinement, the release agent includes ethylenediaminetetraacetic acid.

As set forth above, the transition metal and/or lanthanide metal ions are independently present in an amount of at least 0.01 μM. In some refinements, the transition metal and/or lanthanide metal ions are independently present in an amount of at least 0.01 μM. In some refinements, transition metal and/or lanthanide metal ions are independently present in an amount of at least 0.001 μM, 0.01 μM, 0.05 μM, 1 μM, 2 μM, 3 μM, 5 μM, or 10 μM.

As set forth above, the target solution has a pH from 4 to 9. In some refinements, the target solution has a pH of at least 4, 4.5, 5, 5.5, 6, 6.5, or 7. In further refinements, the target solution has a pH of at most 11, 10, 9.5, 9, 8.5, 8, or 7.7.

In another aspect, the target solution is an aqueous solution that includes a buffer (e.g., a HEPES buffer).

In another embodiment, a method for removing metal ions from a solution is provided. The method includes a step of providing a target solution that includes transition metal ions and/or lanthanide metal ions. The target solution is contacted with a protein-chelator system that includes a chelating moiety having formula I or II or salts thereof conjugated to a protein having at least lysine residue on the surface of the protein. Advantageously, the chelating moiety is conjugated through an amide bond formed from a nitrogen atom in the at least one lysine residue and a carboxylate moiety in the chelating moiety having formula I or III:

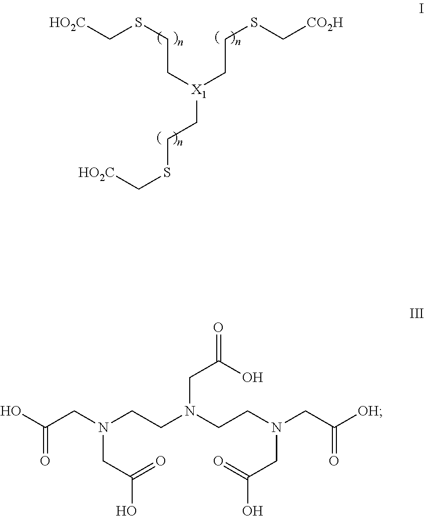

wherein:
X₁ is N or P; and
n is 1 or 2.
   In a refinement, the method includes a step of collecting a precipitate that includes the transition metal ions or lanthanide metal ions. Examples of transition metal ions include Ni ions (e.g., Ni(II)) and/or Cu ions (e.g., Cu(II)). Examples of lanthanide metal ions include europium ions (e.g., Eu(II)). In a refinement, the salt is a sodium or potassium salt.

As set forth above, the method further includes a step of contacting the precipitate with a release agent to recover the transition metal ions or lanthanide metal ions in solution. In a refinement, the release agent includes ethylenediaminetetraacetic acid.

As set forth above, the protein includes a plurality of lysine residues. In a refinement, the protein includes at least 3 lysine residues. In another refinement, the protein includes at least 5 lysine residues. In some refinements, the protein includes at least 1, 2, 3, 4, 5, 6, 7, 8, 9 or 10 lysine residues. In further refinements, the protein includes at most 30, 25, 23, 20, 18, 15, 13, or 10 lysine residues.

As set forth above, the plurality of lysine residues includes at least one lysine residue positioned at the surface of the protein. In some refinements, the plurality of lysine residues includes at least 1, 2, 3, 4, or 5 lysine residues positioned at the surface of the protein. In a refinement, In further refinements, the protein includes at most 15, 13, 10, 8, or 5 lysine residues positioned at the surface of the protein. Although virtually any protein having lysine residues can be used, useful examples include myoglobin and albumin.

As set forth above, the transition metal and/or lanthanide metal ions are independently present in an amount of at least 0.01 μM. In some refinements, the transition metal and/or lanthanide metal ions are independently present in an amount of at least 0.01 μM. In some refinements, transition metal and/or lanthanide metal ions are independently present in an amount of at least 0.001 μM, 0.01 μM, 0.05 μM, 1 μM, 2 μM, 3 μM, 5 μM, or 10 μM.

As set forth above, the target solution has a pH from 4 to 9. In some refinements, the target solution has a pH of at least 4, 4.5, 5, 5.5, 6, 6.5, or 7. In further refinements, the target solution has a pH of at most 11, 10, 9.5, 9, 8.5, 8, or 7.7.

In another aspect, the target solution is an aqueous solution that includes a buffer. In a refinement, the protein-chelator system is soluble in the target solution is an aqueous solution that includes a buffer.

In another embodiment, a method for removing metal ions from a solution is provided. The method includes a step of providing a target solution that includes transition metal ions or lanthanide metal ions. The target solution with a protein-chelator system that includes a chelating moiety having formula II or salts thereof conjugated to a protein having at least lysine residue on the surface of the protein. Advantageously. the chelating moiety is conjugated through an amide bond formed from a nitrogen atom in the at least one lysine residue and a carboxylate moiety in the chelating moiety having formula III:

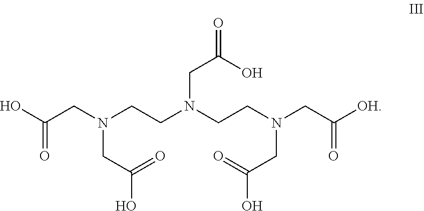 [SMILES: O=C(O)CN(CCN(CC(=O)O)CC(=O)O)CCN(CC(=O)O)CC(=O)O]

In a refinement, the protein-chelator system is soluble in the target solution. Examples of transition metal ions include Ni ions (e.g., Ni(II)) and/or Cu ions (e.g., Cu(II)). Examples of lanthanide metal ions include europium ions (e.g., Eu(II)). In a refinement, the salt is a sodium or potassium salt.

In another aspect, the target solution is a first buffer solution. In a refinement, the first buffer solution is a HEPES buffer.

In another aspect, further includes a step of exchanging the first buffer solution with a second buffer solution in which chelated transition metal an/or lanthanide metal ions are insoluble. In a refinement, the second buffer solution is a phosphate buffer.

As set forth above, the transition metal and/or lanthanide metal ions are independently present in an amount of at least 0.01 μM. In some refinements, the transition metal and/or lanthanide metal ions are independently present in an amount of at least 0.01 μM. In some refinements, transition metal and/or lanthanide metal ions are independently present in an amount of at least 0.001 μM, 0.01 μM, 0.05 μM, 1 μM, 2 μM, 3 μM, 5 μM, or 10 μM.

As set forth above, the target solution has a pH from 4 to 9. In some refinements, the target solution has a pH of at least 4, 4.5, 5, 5.5, 6, 6.5, or 7. In further refinements, the target solution has a pH of at most 11, 10, 9.5, 9, 8.5, 8, or 7.7.

As set forth above, the protein includes a plurality of lysine residues. In a refinement, the protein includes at least 3 lysine residues. In another refinement, the protein includes at least 5 lysine residues. In some refinements, the protein includes at least 1, 2, 3, 4, 5, 6, 7, 8, 9 or 10 lysine residues. In further refinements, the protein includes at most 30, 25, 23, 20, 18, 15, 13, or 10 lysine residues.

As set forth above, the plurality of lysine residues includes at least one lysine residue positioned at the surface of the protein. In some refinements, the plurality of lysine residues includes at least 1, 2, 3, 4, or 5 lysine residues positioned at the surface of the protein. In a refinement, In further refinements, the protein includes at most 15, 13, 10, 8, or 5 lysine residues positioned at the surface of the protein.

Additional details are provided in Sayak Gupta, Cassandra L. Ward, S. Sameera Perera, Conor T. Gowan, Timothy M. Dittrich, Matthew J. Allen, Shawn P. McElmurry, and Jeremy J. Kodanko; Development of a Highly Selective Ni(II) Chelator in Aqueous Solution; Inorganic Chemistry 2022 61 (48), 19492-19501DOI: 10.1021/acs.inorgchem.2c0344 and the supporting materials; Sayak Gupta, Kelli Chapman, Sai Praneeth, Paul M. Stemmer, Matthew J. Allen, Timothy M. Dittrich, and Jeremy J. Kodanko, Semi-Synthetic Proteins as Metal Ion Capture Agents: Catch and Release of Ni(II) and Cu(II) with Myoglobin Bioconjugates; ACS Sustainable Chem. Eng. 2023, 11, 30, 11305-11312, Jul. 20, 2023, https://doi.org/10.1021/acssuschemeng.3c03148 and its supplementary information; the entire disclosures of which are hereby incorporated by reference.

The following examples illustrate the various embodiments of the present invention. Those skilled in the art will recognize many variations that are within the spirit of the present invention and scope of the claims.

### 1. Development of a Highly Selective Ni(II) Chelator in Aqueous Solution

### 1.1 Introduction

In this section, the design, synthesis, and characterization of a highly selective Ni(II) chelator SG-20 is described. Based on lessons learned from NS-1 and NiCast, the aniline amine in NS-1 and NiCast is replaced with a basic amine would result in stronger binding to Ni(II). Therefore, the aromatic groups in NS-1 and NiCast were replaced with a third SO arm, resulting in the C₃-symmetric ligand SG-20. Therefore, a new symmetric ligand is synthesized from the cheap and commercially available starting material triethanolamine. It is expected that not all three carboxylates of SG-20 would bind to the same Ni(II) ion at once, which would leave one side arm free to conjugate the ligand to suitable carriers, such as a solid support, to accomplish Ni(II) capture. The studies set forth below revealed that SG-20 showed greater affinity toward Ni(II) than NS-1 or NiCast. Moreover, SG-20 was selective in binding with Ni(II) compared to other metal ions including Zn(II), Fe(II), Co(II), and, most importantly, Cu(II). Studies revealed that SG-20 binds to Ni(II) at neutral pH with lower affinity upon reducing pH, which might aid in Ni(II) capture and release. X-ray crystallographic analysis of a Ni complex formed from SG-20 revealed that the ligand adopts a NS₂O₂ coordination mode with an axial aqua ligand completing the octahedral donor set, leaving one SO arm free to bind neighboring Ni(II) ions within a cluster. The unique ability of SG-20 to support cluster formation with Ni(II) is expected to be impactful in the future to conjugate SG-20 to suitable carriers for selective removal of Ni(II) from solution.

### 1.2 Experimental Section

1.2.1 Materials. All materials received were used without any further purification unless otherwise specified. Triethanol amine, dimethyl formamide, methyl thioglycolate, thionyl chloride, chloroform, copper(II) chloride dihydrate, and potassium carbonate were purchased from Sigma Aldrich. Phosphoryl chloride, sodium iodide, nickel(II) chloride hexahydrate, cobalt(II) chloride hexahydrate, zinc(II) chloride, and ferrous ammonium sulfate hexahydrate (Fe(NH4)2(SO4)2) were purchased from Fischer Scientific. All deuterated solvents were purchased from Cambridge Isotope Laboratories. Compound 1 was prepared following a reported procedure.^{22,23}

1.2.2 Instrumentation. NMR spectra were recorded using a Varian FTNMR Mercury 500 MHz spectrometer. The electronic absorption spectra were collected using a Carey 60 spectrophotometer. The infrared (IR) spectra were taken using a Bruker Tensor 27 Fourier transform infrared (FT-IR) spectrometer.

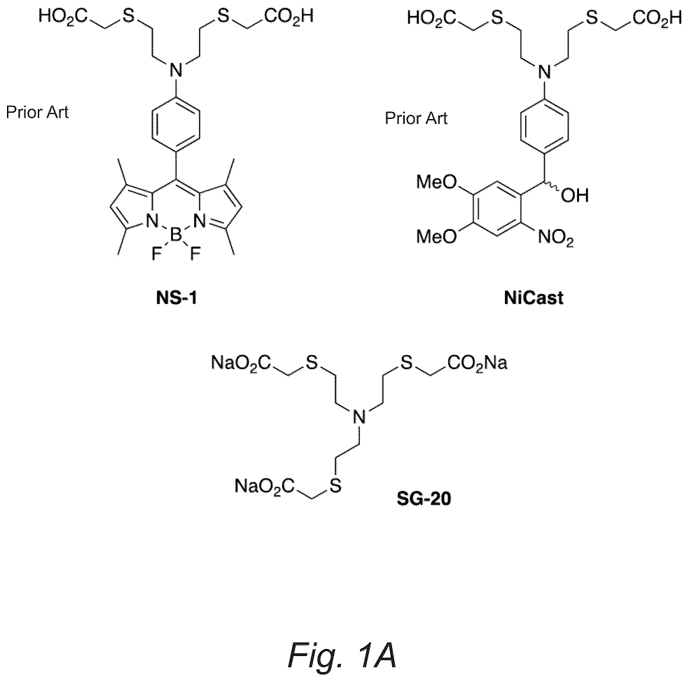

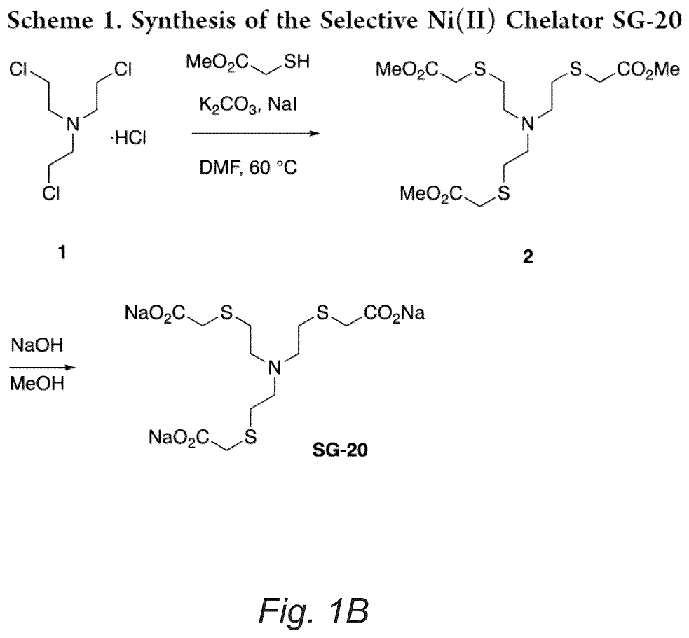

1.2.3 Synthesis of Trimethyl 2,2′,2″-((Nitrilotris(ethane-2,1-diyl))-tris(sulfanediyl))triacetate (2). The synthetic scheme for this preparation is found in FIG. 1B. A solution of 1 (3.0 g, 15 mmol) in anhydrous dimethylformamide (150 mL) was treated with K₂CO₃ (12.2 g, 88.2 mmol) and NaI (1.1 g, 7.4 mmol). Methyl thioglycolate (5.8 mL, 74 mmol) was added dropwise, and the mixture was then heated at 60° C. for 24 h under an atmosphere of N₂. After 24 h, the reaction mixture was poured in water (200 mL) and extracted with ethyl acetate (2×150 mL). The organic layers were combined, dried with MgSO₄, and concentrated under reduced pressure to yield a yellow oil. The resulting product was dissolved in tert-butyl methyl ether (100 mL), washed with water (2× 100 mL), dried with MgSO₄, and concentrated under reduced pressure. The crude product was purified using column chromatography (silica gel, 2-4% methanol in dichloromethane) to yield compound 2 as a colorless oil (2.12 g, 35%). ¹H NMR (500 MHz, CDCl3): δ 2.75 (m, 12H), 3.26 (s, 6H), 3.74 (s, 9H). 13C NMR (125 MHz) in CDCl3 δ=30.3, 33.5, 52.6, 55.6, 170.9 ppm. ESI-MS(+): [M+H]+m/z=observed: 414.11, calculated: 414.11. IR (KBr, cm⁻¹) v(C═O) 1733 s.

1.2.4 Synthesis of Sodium 2,2′,2″-((Nitrilotris(ethane-2,1-diyl))-tris(sulfanediyl))triacetate (SG-20). An aqueous solution of NaOH (10 M, 0.21 mL, 2.1 mmol) in water was added to a solution of compound 2 (292 mg, 0.7 mmol) in methanol (30 mL). The resulting solution was stirred for 18 h at ambient temperature and then concentrated to dryness under reduced pressure to furnish SG-20 as a white solid (253 mg, 82%). ¹H NMR (500 MHz, D20): δ 2.58 (t, 6H, J=5 Hz), 2.67 (t, 6H, J=5 Hz), 3.11 (s, 6H). 13C NMR (125 MHz) in D2O δ=28.3, 36.8, 52.4, 178.1 ppm. ESI-MS(+): [SG-20-3Na+4H]+m/z=observed: 372.06, calculated: 372.06, [SG-20−2Na+3H]+m/z=observed: 394.04, calculated: 394.05, [SG-20-Na+2H]+m/z=observed: 416.02, calculated: 416.03, [SG-20+H]+m/z=observed: 438.01, calculated: 438.01, [SG-20+Na]+m/z=observed: 459.98, calculated: 459.99. IR (KBr, cm⁻¹) vₐₛ(COO⁻) 1580 s, vₛ(COO⁻) 1397 s.

1.2.5 Synthesis of Sodium 2,2′,2″-((Nitrilotris(ethane-2,1-diyl))-tris(sulfanediyl))triacetate-Ni(II) Adduct (Ni-SG-20). A mixture of NiCl₂·6H₂O (11 mg, 0.05 mmol), SG-20 (20 mg, 0.05 mmol), and methanol (20 mL) was stirred, transforming from a heterogeneous mixture into a homogeneous yellow/green solution within 3 h. After 3 h, the solution was dried under reduced pressure. The resulting solid was dissolved in a minimal amount of methanol (˜3 mL). Vapor diffusion of diethyl ether into the methanol solution resulted in a greenish powder that was isolated by filtration (5 mg). IR (KBr, cm⁻¹) vₐₛ(COO⁻) 1616 s, vₛ(COO⁻) 1461 s. (Electronic absorption, H₂O) λₘₐₓ (nm) 260. ESI-MS(+): [SG-20+Ni⁵⁸−3Na+2H]+m/z=observed: 427.95, calculated: 427.98, [SG-20+Ni⁵⁸−2Na+H]+m/z=observed: 449.90, calculated: 449.95, [SG-20+Ni⁶⁰−2Na+H]+m/z=observed: 451.90, calculated: 451.95, [SG-20+Ni⁵⁸−Na]+m/z=observed: 471.95, calculated: 471.94.

1.2.6 Job Plot Studies. Solutions of NiCl₂·6H₂O or CuCl₂·2H₂O and ligand SG-20 were prepared in 4-(2-hydroxyethyl)-1-piperazineethanesulfonic acid (HEPES) buffer (pH=7.1). The mole fractions for each of the metal ions varied from 0 to 1, and the total concentration of the solutions were maintained at 25 μM for Ni(II) and 125 μM for Cu(II). Electronic absorption spectra were acquired, and absorbance changes at λₘₐₓ [260 nm for Ni(II) and 350 nm for Cu(II)] relative to free SG-20 versus mole fraction of the metal ion were plotted. Two best-fit lines were drawn which intersected at a mole fraction of ˜0.5, indicating 1:1 binding of SG-20 to Ni(II) and Cu(II).

1.2.7 Determination of K_{d} Values. K_{d} values were measured in HEPES buffer (pH=7.1) for Cu(II) and Zn(II) and in acetate buffer (pH=5.6) for Co(II) and Fe(II), owing to the limited solubility of those ions in HEPES buffer. K_{d} values for Ni(II) were measured in both HEPES and acetate buffers for direct comparison. Solutions of SG-20 [5 and 25 μM for Ni(II) and 25 μM for Cu(II) and Fe(II)] were treated with solutions of NiCl₂·6H₂O, CuCl₂·2H₂O, or FeSO₄·(NH₄)2SO₄·6H₂O and monitored by electronic absorption spectroscopy. New peaks formed [λ=260 nm for Ni(II), λ=280 nm for Fe(II), and λ=350 nm for Cu(II)]. Changes in absorbance versus concentration of metal ions were plotted, and data were fit to the Hill equation using Graphpad Prism to determine K_{d} values. Co(II) and Zn(II) were spectroscopically silent. Therefore, to determine K_{d} values for Zn(II) and Cu(II), solutions of SG-20 and NiCl₂·6H₂O (25 μM, 1:1) were titrated against CoCl₂·2H₂O or ZnCl₂·2H₂O. The decrease in the absorbance at λ=260 nm was plotted against the concentration of Zn(II) or Co(II).

1.2.8 Effect of pH on Binding of SG-20 with Ni(II). A solution of SG-20 and Ni(II) (25 μM, 1:1) was prepared in HEPES buffer (20 mM, 20 mL, pH=7.1). Aliquots of aqueous solution of HCl (1.0 M, 0-1.1 mL) were added, and pH was monitored. Aliquots of the solution were taken and monitored by electronic absorption spectroscopy. The addition of HCl was continued until the characteristic peak at λ=260 nm diminished.

1.2.9 Determination of the pKₐ Values of SG-20. SG-20 (30 mg, 0.7 mmol) was dissolved in 0.1 M HCl containing 0.1 M KNO3 (10 mL). The solution was titrated against 0.5 M NaOH. The pH of the solution was recorded on addition of NaOH (0-14 mL) until the pH reached ˜12. The titration curve (pH vs V) and the first differential curve (ΔpH/ΔV vs V) were plotted, and the pKₐ values were determined from the corresponding inflection points in the first differential curve.

1.2.10 X-ray Photoelectron Spectroscopy Analysis. Powder samples of SG-20 and Ni-SG-20 were analyzed using the ThermoScientific Nexsa X-ray photoelectron spectrometer with a hemispherical analyzer and monochromatic Al Kα source. Powder samples were mounted onto a sample holder using the Cu tape. A thick layer of powder (˜1 mm) was adhered to the Cu tape to ensure that all signals were from the sample. Then, the sample holder was loaded into the entry-lock and held under vacuum (<5×10-6 mbar) for ˜30 min. Once the pressure in the entry-lock chamber reached ˜4×10⁻⁷ mbar, the sample holder was transferred to the analysis chamber. The base pressure of the analysis chamber during the data acquisition was ˜2.1×10⁻⁷ mbar.

X-ray photoelectron spectroscopy (XPS) survey spectra were collected using a pass energy of 150 eV, an energy step size of 1.0 eV, and a dwell time of 10 ms/step. After the survey scan, high-resolution spectra of C 1s, O 1s, S 2p, Na 1s, and Ni 2p core lines were collected using a pass-energy of 50 eV, an energy step size of 0.1 eV, and a dwell time of 100 ms/step. Spectra were analyzed using ThermoAvantage v5.9922 software to extract qualitative and quantitative information from the survey and high-resolution scans. The peaks were fitted using a combination of Gaussian and Lorentzian functions.

1.2.11 X-ray Crystallography. Single crystals suitable for X-ray diffraction (XRD) were grown according to a modified literature procedure for Ni(II) and nitrilotripropionic (NTP) acid.²⁴ Ligand SG-20 (20 mg, 0.05 mmol) and NiCl₂·6H₂O (11 mg, 0.05 mmol) were added to H₂O (3 mL) followed by stirring and filtration of the reaction mixture. Slow evaporation of the solvent over 2 weeks resulted in formation of green crystals that were analyzed by electronic absorption spectroscopy in water (λₘₐₓ=260 nm). One crystal (0.03×0.050×0.08 mm3) was mounted on a MicroMount (MiTeGen) with paratone oil (Parabar 10312, Hampton Research) on a Bruker D8 Venture diffractometer with kappa geometry, an Incoatec IμS micro-focus source X-ray tube (Cu Kα radiation), and a multilayer mirror for monochromatization. XRD intensities were measured using a Photon III CPAD area detector at a distance of 40 mm and 0.5° image width. Data were acquired at 100 K with an Oxford 800 Cryostream low-temperature apparatus. The intensities were integrated using SAINT V8.38a. A multiscan absorption correction was applied with SADABS v2016/2 using APEX4 v2021.10-0. The crystal structure was solved using a dual-space approach as implemented in SHELXT and difference Fourier (ΔF) maps during least-squares refinement, as embedded in SHELXL-2019 running under Olex2.²⁵⁻²⁷ All non-hydrogen atoms were refined anisotropically. Hydrogen atoms were positioned with idealized geometry and refined isotropically using a riding model. The complex crystallized in the centrosymmetric space group C2/c with one cluster in the asymmetric unit and Z=8. A solvent mask was applied using Olex2 to remove the disordered solvated water molecules. Since a solvent mask was applied to remove the remaining disordered water, the hydrogen bonding network could not be determined for the waters that were included in the crystal structure; hence, there are several B alerts for the lack of hydrogen bond acceptors. The remaining B-alerts are due to a large density near the solvated Na ion which is likely due to partially occupied water.

### 1.3 Results

1.3.1 Synthesis, pKₐ determination, and PHREEQC Modeling of SG-20. Our designed ligand, SG-20, contains one basic nitrogen, three sulfur donor atoms, and three carboxylate groups, resulting in the ability to bind metal ions via a number of modes, including monodentate, bidentate, or bridging.²⁸ The ligand was isolated as a trisodium salt in three steps starting from triethanolamine. Briefly, triethanolamine was refluxed in SOCl₂ to provide 1.^{23,29} The second step involved nucleophilic displacement of the three chlorides of 1 with methyl thioglycolate, where 1 was heated to 60° C. under an atmosphere of N₂ in presence of K₂CO₃ (6 equiv) and catalytic NaI (0.5 equiv), resulting in 2 with an isolated yield of 35% after silica gel chromatography (Scheme 1). Finally, trimethyl ester 2 was saponified in the presence of NaOH (3.0 equiv) in methanol at ambient temperature.²⁹ SG-20 was obtained as a white solid in 82% yield.

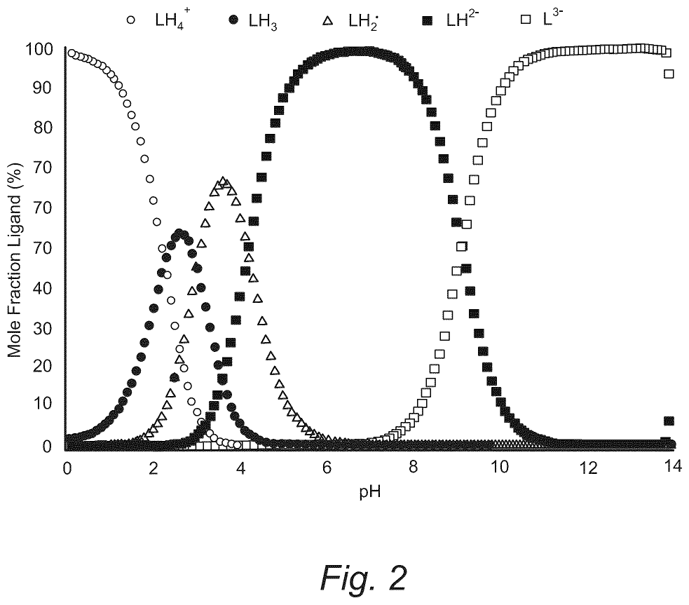

The pKₐ values of SG-20 were measured by titrating a solution of SG-20 in 0.1 M HCl against NaOH. A constant ionic strength was maintained using KNO3 (0.1 M).^{30,31} The first differential of the titration curve showed four sharp inflection points corresponding to the four pKₐ values of SG-20. The pKₐ1, pKₐ2, pKₐ3, and pKₐ4 values of SG-20 were determined to be 2.29, 3.07, 4.33, and 9.33, respectively. These pKₐ values are in good agreement with values reported for NTP acid whose structure is similar to that of SG-20.³² Based on the calculated pKₐ values of SG-20 (denoted as L in FIG. 2), the major chemical species prevalent in solution were determined with the help of PHREEQC modeling. From our PHREEQC calculations, it is evident that at low pH (pH=0-2), the major species in solution is LH₄⁺. However, upon increasing the pH of the solution, the mole fraction of LH₄⁺ decreases and that of LH₃ increases. The mole fraction of LH3 reaches a maximum at pH ˜2.5. Upon further increase in pH, the major species in solution was observed to be LH2− that reaches its maximum mole fraction at a pH value of ˜3.8. When the pH increases above 4, the major species present in solution is LH2−, which reaches at maximum at a pH value of ˜7.0. The mole fraction of LH2− decreases with increasing pH, and L3− becomes the major species in solution as the pH of the solution increases above 9.

1.3.2 Binding Studies of SG-20 with Different Metal Ions. Dissociation constants (K_{d}) indicate the strength of binding of a ligand to a metal ion and can be used to determine the selectivity of a ligand toward various metals.³³-35 The constant K_{d} is the concentration at which 50% of the metal ion is bound to ligand. To determine the K_{d} of the ligand, SG-20 was titrated at a fixed concentration (25 μM) against varying concentrations of Ni(II), Cu(II), Zn(II), Fe(II), and Co(II), and the titrations were monitored by electronic absorption spectroscopy. Titration experiments were performed in HEPES buffer (pH=7.1) for Ni(II), Cu(II), and Zn(II) and in acetate buffer (pH=5.6) for Fe(II) and Co(II). The titrations were performed in acetate buffer for Fe(II) and Co(II), owing to their limited solubility in HEPES buffer, and were conducted under an atmosphere of N₂ to avoid aerobic oxidation of the metal ions. Titration experiments of Ni(II) were also performed in acetate buffer (pH=5.6) to determine the selectivity of SG-20 toward Ni(II) versus Fe(II).

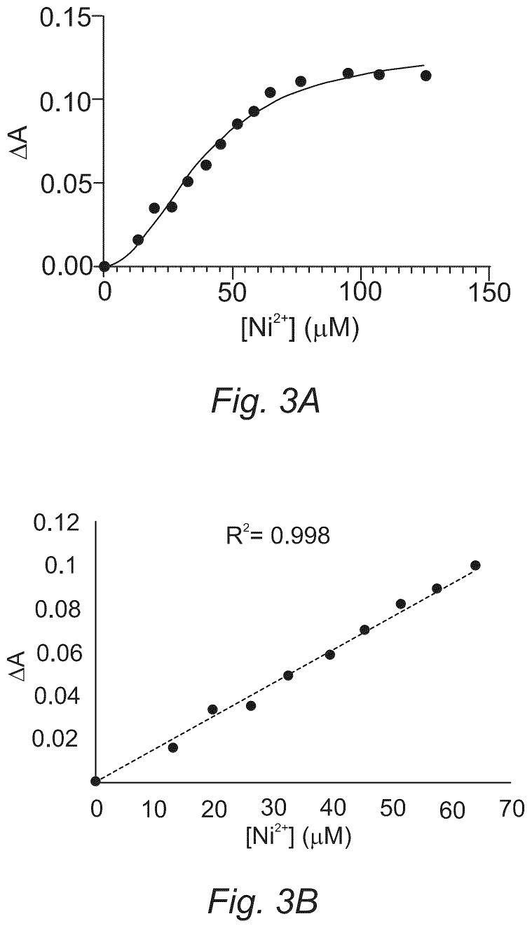

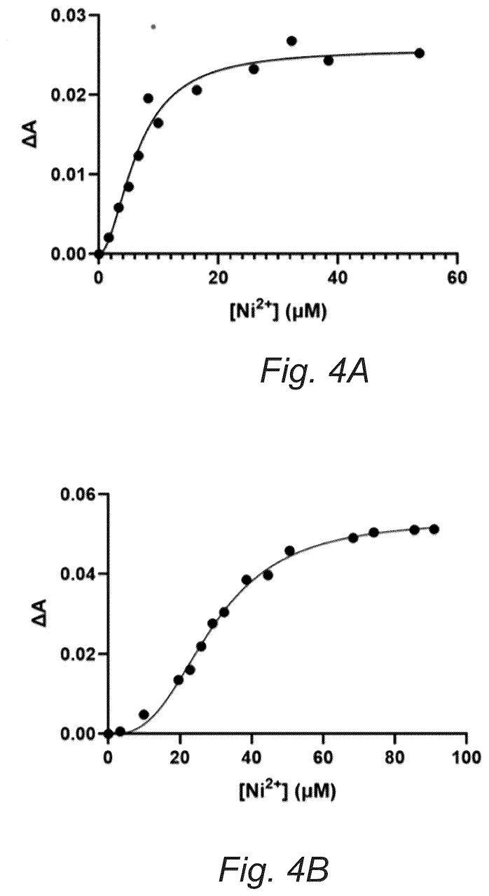

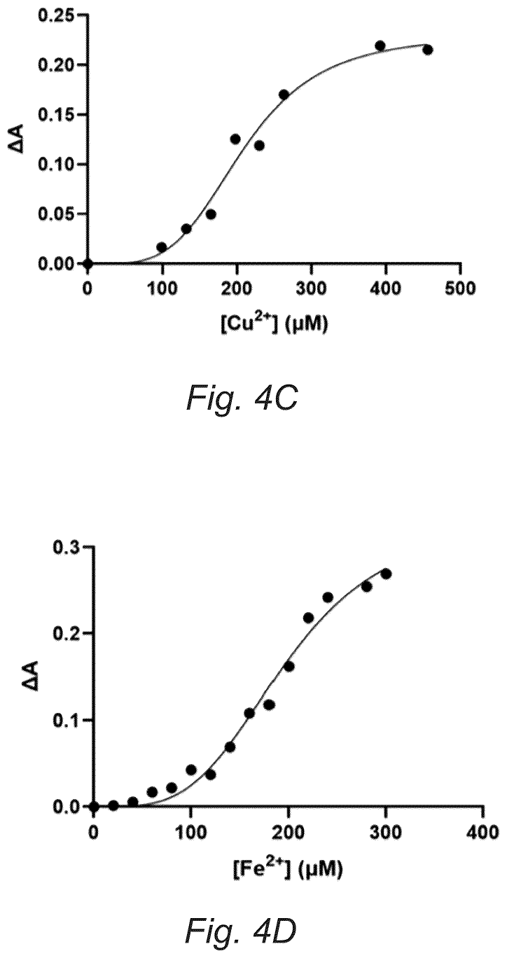

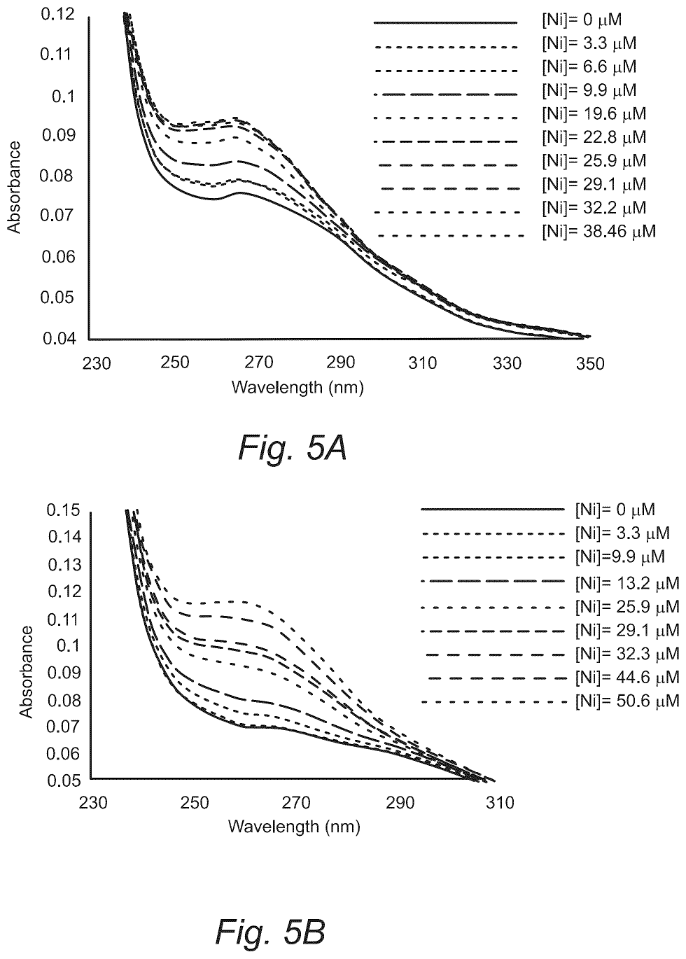

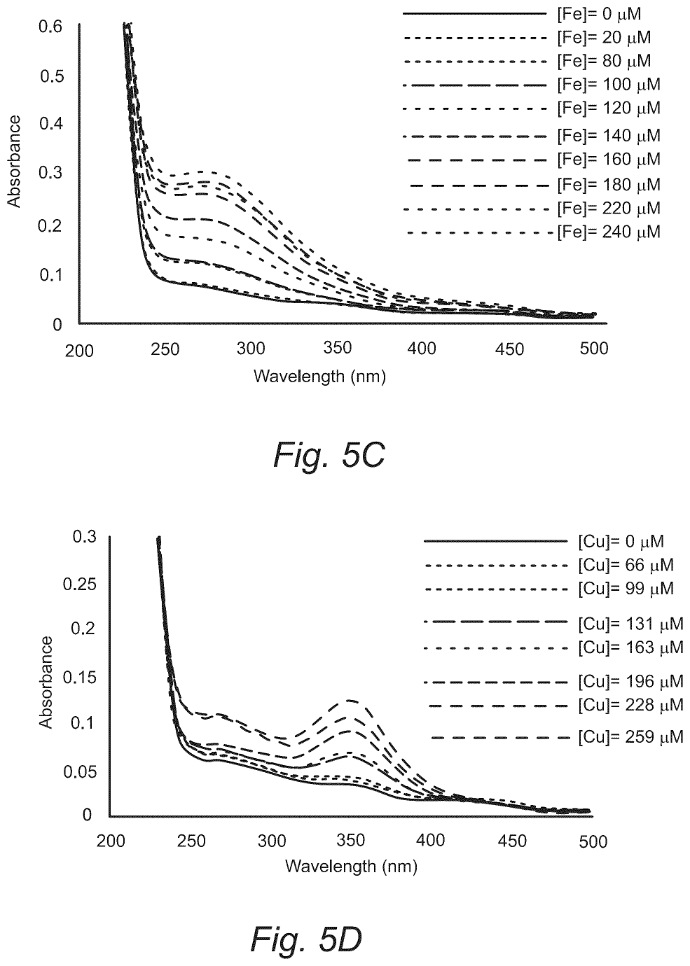

Titration of SG-20 (25 μM) against Ni(II) showed the formation of a new peak at λₘₐₓ=260 nm, consistent with a mixed N, S, and O atom donor set binding to Ni(II),^{36,37} which reached a maximum absorbance value above ˜75 μM Ni(II) (FIG. 3A). Attempts at fitting the change in absorbance (ΔA) versus concentration of Ni(II) to the Hill equation resulted in an unsatisfactory fit, with initial points in the curve being highly linear (R2=0.998, FIG. 3B). Linearity in the initial points suggests that Ni(II) binding is occurring in the titration regime, which can lead to inaccurate determinations of K_{d}.³⁸ Reducing the concentration of ligands in a titration experiment is one way to move outside the titration regime and determine accurate K_{d} values. Upon reducing the concentration of SG-20 to 5 μM, a satisfactory fit to the Hill equation was obtained, resulting in a K_{d} value for Ni(II) of 7.0±0.4 μM (FIG. 4A and Table 1). Ni(II) was also titrated against SG-20 (25 μM) in acetate buffer (pH=5.6), which gave a satisfactory fit to the Hill equation, resulting in a K_{d} value of 25=3 μM (FIG. 4B and Table 1). The larger K_{d} value of SG-20 against Ni(II) in acetate buffer versus HEPES buffer indicates weaker binding of SG-20 toward Ni(II) at lower pH (vide infra). Titration experiments for Cu(II) and Fe(II) with SG-20 (25 μM) showed the formation of new peaks at λₘₐₓ=350 and 280 nm, respectively, with similar saturation of absorbance behavior (FIG. 5). Fitting the data using the Hill equation revealed satisfactory fits with positive cooperativity (FIG. 4C,D).³⁹ The K_{d} values were found to be 200±7 and 160±30 μM for Cu(II) and Fe(II), respectively. Thus, SG-20 was 29 and 6.5 times more selective in binding Ni(II) than Cu(II) and Fe(II), respectively. Importantly, SG-20 was selective for Ni(II) over Cu(II), unlike NS-1 and NiCast.^{20,21} Titration of SG-20 (25 μM) with Zn(II) and Co(II) did not lead to absorbance changes versus free metal ions [Zn(II) was spectroscopically silent]. Thus, K_{d} values for Zn(II) and Co(II) were estimated using competition experiments. Solutions of SG-20 and Ni(II) (1:1, 25 μM) were treated with Zn(II) and Co(II), and absorbance was monitored at 260 nm. However, significant decreases in the absorbance at 260 nm were not observed even after concentrations of Zn(II) and Co(II) reached 300 and 456 μM, respectively. We therefore concluded that the apparent K_{d} of SG-20 for Zn(II) was >>300 μM and that for Co(II) was >>456 μM. Overall, these studies reveal that SG-20 binds with strong affinity to Ni(II) and high selectivity versus other first-row transition metals that are abundant components of coal fly ash extracts. Studies for K_{d} determination suggested binding of Ni(II), Cu(II), and Fe(II) with SG-20. Based on these results, binding stoichiometries of SG-20 with Ni(II), Cu(II), and Fe(II) were determined using Job plot analysis.⁴⁰⁻⁴⁴ Job plot analysis is a method in which the mole fractions of the ligand and metal ion varied, but the total concentrations of each solution are held constant. The mole fraction at which the maximum absorbance is obtained reveals the stoichiometry of binding in solution. Data revealed that maximum absorbance of solutions containing SG-20 and Ni(II) was obtained when the mole fraction of Ni(II) in the solution was 0.48 (˜0.5) at λ=260 nm. Similarly, for solutions containing SG-20 and Cu(II), the maximum absorbance was obtained for mole fraction of 0.45 (˜0.5) at λ=350 nm. These results are consistent with Ni(II) and Cu(II) binding with SG-20 with a 1:1 stoichiometry. The results of the Job plot analysis of Ni(II) and SG-20 were verified using ESI mass spectrometry. The ESI mass spectra of a solution of NiCl₂·6H₂O and SG-20 (1:1) in H₂O showed peaks at m/z=427.90, 449.75, 451.85, and 471.90 for the species [SG-20+Ni⁵⁸−3Na+2H]+, [SG-20+Ni⁵⁸−2Na+H]+, [SG-20+Ni⁶⁰−2Na+H]+, and [SG-20+Ni⁵⁸−Na]+, respectively. These results are consistent with the formation of a mononuclear complex between Ni(II) and SG-20 in solution. Job plots with Fe(II) and SG-20 were attempted, but data remained inconclusive, due to shifts in Amax values across different mole fractions, which might indicate complex speciation in solution.

TABLE 1
K_{d} Values^{a} of Different Metal Ions against L along with
the Corresponding Ni(II) Selectivity Values, Kd (Ni(II))/Kd
(M(II)) (M(II) = Cu(II), Fe(II), Zn(II), and Co(II))

| | Kd (μM) in | Kd (μM) in | Ni(II) | Ni(II) |
|---|---|---|---|---|
| Metal | HEPES | acetate | selectivity in | selectivity in |
| ion | buffer | buffer | HEPES buffer | acetate buffer |
| Ni(II) | 7.0 ± 0.4 | 25 ± 3 | | |
| Cu(II) | 200 ± 7 | nd | 29 | |
| Fe(II) | nd | 160 ± 30 | | 6.5 |
| Zn(II) | >>300^{b} | nd | >>43 | |
| Co(II) | nd | >>456^{b} | >>19 | |

^{a}Data are reported as the average of three independent experiments, with errors as standard deviations.
^{b}Kd values could not be determined for Zn(II) and Co(II), no new peaks were observed; significant decreases in the absorbance at λ = 260 nm in a solution of SG-20 and Ni(II) (25 μM, 1:1) were not observed upon addition of excess Zn(II) and Co(II);
nd = not determined.

The binding studies indicate that SG-20 shows strong affinity for Ni(II) at neutral pH (˜7.1). To explore potential applications of SG-20 toward Ni(II) capture and release, the effects of pH on Ni(II) binding were determined. Solutions of SG-20 and Ni(II) (1:1, 25 μM) were titrated with HCl (1.0 M), and absorbance at λ=260 nm was monitored, ensuring minimal dilution of the solution. A gradual decrease in absorbance at λ=260 nm was observed between pH 7 and 5, dropping rapidly at pH 4.3. Further lowering the pH of the solution to 3.74 resulted in complete disappearance of the characteristic peak at λ=260 nm, consistent with SG-20 no longer being bound to Ni(II). Overall, the pH dependence of Ni(II) binding is consistent with protonation of the ligand carboxylates, facilitating the release of Ni(II). This behavior is likely to be useful for Ni(II) capture and release once SG-20 is conjugated to a suitable support.

Next, we sought to characterize the nature of the Ni(II) SG-20 complex. Previous studies with NS-1 and NiCast characterized electronic absorption spectral features consistent with Ni(II) binding. However, crystals suitable for XRD studies were not obtained, and some questions remained regarding the coordination number (5 or 6), geometry (square pyramidal or octahedral), and speciation of the complex in solution. Given that evidence of aggregation was observed with Zn(II) and NiCast, it was possible that the NS₂O₂ donor sets of NS-1 and NiCast did not lead to mononuclear Ni(II) complexes but rather larger oligomeric species in solution. To investigate, treatment of SG-20 (1.0 equiv) with Ni(II) (1.0 equiv) in methanol resulted in the formation of a homogeneous light-green solution after stirring. A greenish solid (Ni-SG-20) was obtained after diffusion of diethyl ether into the solution. This solid, denoted as Ni-SG-20, was analyzed by IR spectroscopy, and the data reveal a shift of the symmetric and asymmetric CO stretch between SG-20 and Ni-SG-20. The difference between the asymmetric and stretching CO frequencies in the Ni-SG-20 was 155 cm⁻¹, consistent with carboxylate binding to Ni(II).^{45,46} Moreover, the electronic absorption spectrum of Ni-SG-20 in H₂O showed the same characteristic peak at λ=260 nm that was observed during our titration experiments with SG-20 and Ni(II). The ESI mass spectra of Ni-SG-20 in H₂O showed peaks at m/z=427.95, 449.90, 451.90, and 471.95 for the species [SG-20+Ni⁵⁸−3Na+2H]+, [SG-20+Ni⁵⁸−2Na+H]+, [SG-20+Ni⁶⁰−2Na+H]+, and [SG-20+Ni⁵⁸−Na]+, respectively, suggesting that Ni-SG-20 is a mononuclear complex of Ni(II) and SG-20. To further probe the mode of binding between SG-20 and Ni(II), XPS analysis of SG-20 and Ni-SG-20 was performed.

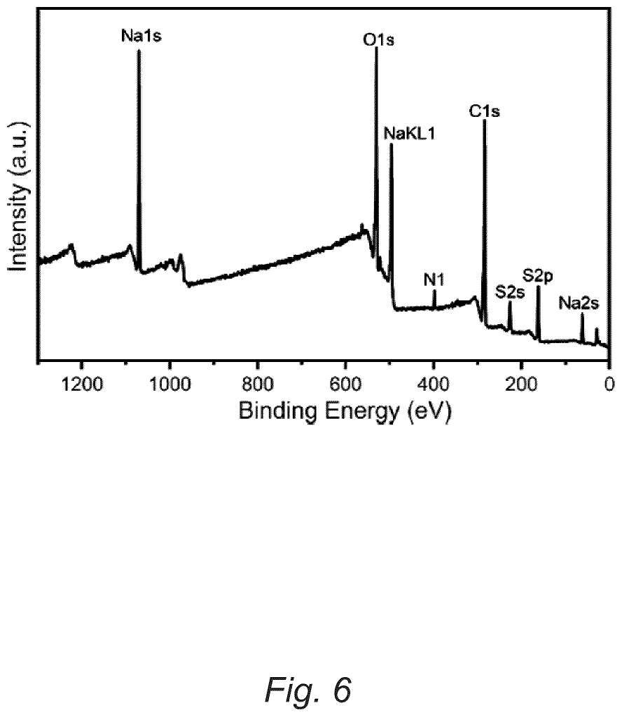

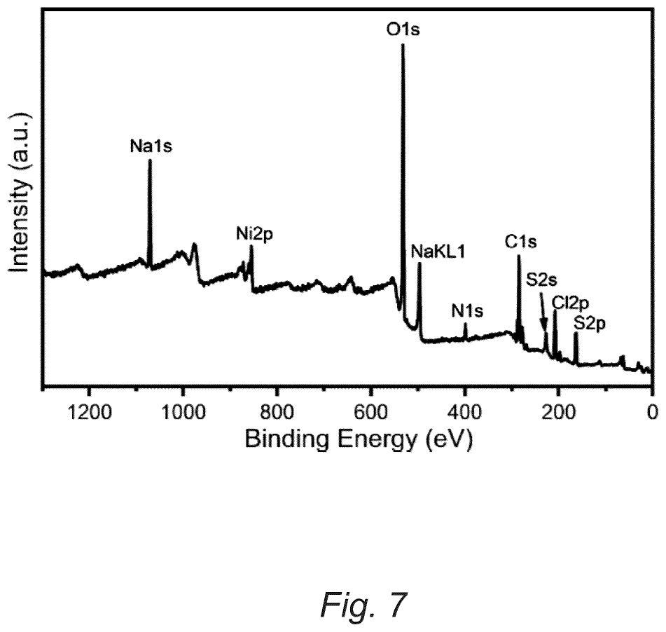

1.3.3 XPS Studies. Determination of the nature of binding of SG-20 with Ni(II) was carried out by comparing the survey spectrum (FIGS. 6 and 7) and binding energies (Tables 2 and 3) of SG-20 and Ni-SG-20 obtained from XPS. Peak fits to high resolution were carried out for the C 1s, O 1s, S 2p, Na 1s, N 1s, and Ni 2p core lines of the XPS spectra recorded for SG-20 and Ni-SG-20. The XPS spectrum obtained by analysis of Ni-SG-20 reveals the emergence of a Ni2p core line that is not observed in the XPS spectrum of SG-20. Moreover, the binding energies obtained in the Ni 2p core line reveal that Ni is bound to N and O donor atoms.

XPS studies of Ni-SG-20 provided evidence of Ni being bound to the nitrogen and carboxylate groups of SG-20. However, it remained inconclusive from these studies whether the sulfur donors of SG-20 were bound to Ni in Ni-SG-20. Intrigued by our XPS data, we sought to characterize the complex being formed between Ni(II) and SG-20. Multiple attempts to crystallize Ni-SG-20 were unsuccessful, including vapor diffusion and slow evaporation with Ni-SG-20 from solvents including methanol and acetonitrile. However, single green crystals suitable for X-ray crystallographic analysis were obtained when a 1:1 solution of SG-20 and Ni(II) in H2O (see the Experimental Section for details) was allowed to evaporate slowly over 2 weeks at ambient temperature.

TABLE 2
Compositional Information Extracted from the Peak Fits to the
High-Resolution Scans Recorded for the Powder Sample SG-20

| element/core line | binding energy (eV) | chemical species |
|---|---|---|
| | 284.8 | C—C |
| | 286.0 | C—O—C |
| | 288.0 | O—C═O |
| | 289.7 | C═O |
| O 1s | 531.1 | C—O |
| | 533.5 | C═O |
| | 535.5 | Na_{KLL} |
| S 2p | 161.6 | metal sulfides |
| | 162.7 | |
| | 162.9 | C—S |
| | 164.2 | |
| Na 1s | 1070.9 | sodium salts |
| N 1s | 398.9 | C—N |
| Ni 2p | N/A | N/A |

TABLE 3
Compositional Information Extracted from the Peak Fits to
the High-Resolution Scans Recorded for the Sample Ni-SG-20.

| element/core line | binding energy (eV) | chemical species |
|---|---|---|
| carbon (1s) | 284.8 | C—C |
| | 285.9 | C—O—C |
| | 288.4 | O—C═O |
| O 1s | 531.8 | C—O |
| | 533.1 | C═O |
| | 536.6 | |
| | 162.2 | metal sulfides |
| | 163.3 | |
| | 163.9 | C—S |
| | 165.1 | |
| Na 1s | 1070.9 | sodium salts |
| N 1s | 399.0 | C—N |
| | 402.5 | C—N^{δ+} or N—Si |
| Ni 2p | 855.5 | Ni(II)_{Ni—N} |
| | 872.9 | |
| | 857.2 | Ni(II)_{Ni—O} |
| | 874.6 | |
| | 860.4 | Ni(II)_{Sat (Ni—N)} |
| | 877.3 | |
| | 861.9 | Ni(II)_{Sat (Ni—O)} |
| | 879.3 | |
| | 864.2 | Ni(II)_{Sat} |
| | 881.4 | |
| | 866.3 | Ni(II)_{Sat} |
| | 883.4 | |

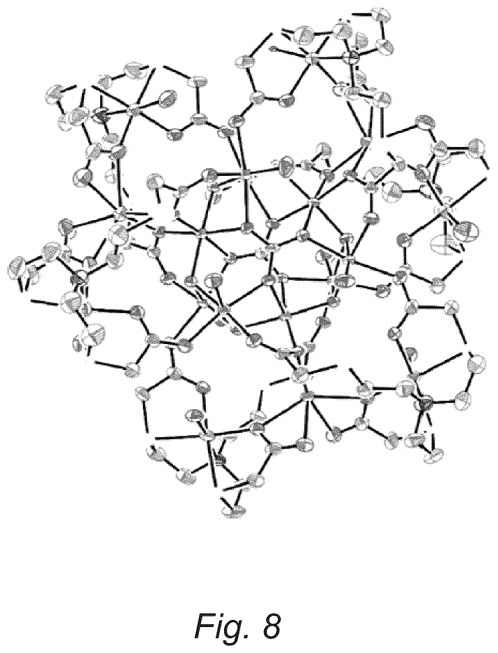

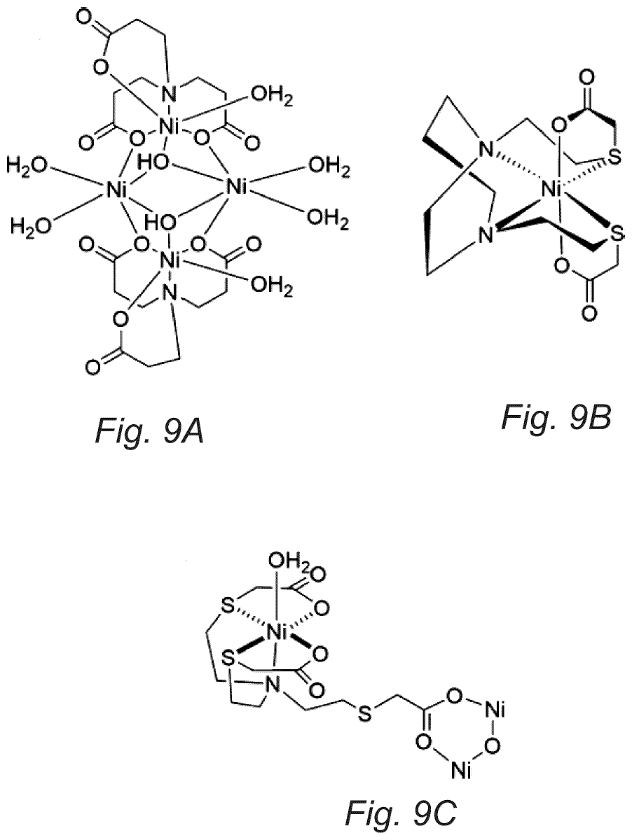

1.3.4 X-ray Crystallography. X-ray crystallographic analysis reveals that Ni(II) and SG-20 formed a Ni(II) cluster containing six SG-20 ligands, 15 Ni(II) centers, three Na(I) centers, and one additional solvated Na(I) ion (NiClust-SG-20). The unit cell of NiClust-SG-20 is monoclinic and belongs to the C2/c space group (Table 4). All Ni(II) centers in the cluster are octahedral. The inner core contains a carbonate group and nine Ni(II) centers containing oxo bridges and carboxylate bridges, and the outer core contains six Ni(II) centers with a S₂NO₃ atom donor set. The source of the central carbonate group within the cluster is likely atmospheric CO2, which helps explain the long time required for the formation of crystals of this complex. The S₂NO₃ core included two S, one N, and two O atoms from SG-20 and the other O atom from a bound H₂O molecule. All of the Ni(II) centers show the coordinated H₂O ligand axial and trans to the basic N atom (FIG. 8). From the structure of NiClust-SG-20, it is clear that two SO chelator arms in SG-20 are involved in binding with one Ni(II) atom. The other SO-containing arm does not bind to the same Ni(II) atom as the other two arms but instead aids in binding with other Ni(II) atoms within the central cluster (see FIG. 9C). Analysis of the NiClust-SG-20 crystals by electronic absorption spectroscopy revealed the same spectral feature that was observed in titration experiments with λₘₐₓ=260 nm. In addition, minor peaks at 310 and 360 nm were also observed that are consistent with other Ni(II) complexes with similar donor sets.⁴⁷⁻⁴⁹ It has been reported that another Ni(II) complex, Ni-1′-Ac₂, with a similar chemical environment like the NS₂O₃ core present in NiClust-SG-20 showed a characteristic peak at λ=258 nm.³⁷

TABLE 4
Crystal Data and Structure Refinement for NiClust-SG-20

| empirical formula | C₇₄H₁₃₂N₆Na₄Ni₁₅O₆₀S₁₈ |
|---|---|
| formula weight | 3615.54 |
| temperature/K | 100.00 |
| crystal system | monoclinic |
| space group | C2/c |
| a/Å | 67.1837(13) |
| b/Å | 25.6597(5) |
| c/Å | 20.4721(4) |
| α/deg | 90 |
| β/deg | 92.6200(10) |
| γ/deg | 90 |
| volume/Å³ | 35255.2(12) |
| Z | 8 |
| ρ_{calc}g/cm³ | 1.362 |
| μ/mm⁻¹ | 4.344 |
| F(000) | 14800.0 |
| crystal size/mm³ | 0.08 × 0.05 × 0.03 |
| radiation | Cu Kα (λ = 1.54178) |
| 2θ range for data | 2θ range for data |
| collection/deg | collection/deg |
| index ranges | −78 ≤ h ≤ 80, −30 ≤ |
| | k ≤ 30, −23 ≤ l ≤ 24 |
| reflections collected | 231,507 |
| independent reflections | 32,379 [Rᵢₙₜ = 0.1326, |
| | R_{sigma} = 0.0737] |
| data/restraints/parameters | 32,379/378/1594 |
| goodness-of-fit on F² | 1.035 |
| final R indexes [I ≥ 2σ (I)] | R₁ = 0.0653, wR₂ = 0.1649 |
| final R indexes [all data] | R₁ = 0.0908, wR₂ = 0.1790 |
| largest diff. peak/hole/e Å⁻⁻³ | 3.33/−0.60 |

The structure of NiClust-SG-20 showed that the Ni—O bond lengths varied from 1.977(3) to 2.140(4) Å, which was consistent with the structure having different types of Ni—O bonds, including oxo and carboxylate bridges. The Ni—N and Ni—S bond lengths varied from 2.120(6) to 2.167(7) Å and 2.387(2) to 2.419(2) Å, respectively. The bond lengths observed in NiClust-SG-20 are consistent with the Ni—O and Ni—N bond lengths in another Ni cluster ([Ni₄(μ₃—OH)₂(H₂O)₆(NTP)₂]) formed between Ni(II) and NTP, a ligand structurally similar to SG-20 (FIG. 9A) which were reported to be 2.007(1) to 2.107(1) Å and 2.065(1) Å, respectively.24 Interestingly, the complex formed between N,Nbis [2-(carboxylmethyl)thioethyl]amine (CTEA) and Cu(II) [Cu(CTEA)] reported Cu—O bond lengths from 1.958(5) to 1.961(5) Å and Cu—S bond lengths from 2.349(6) to 2.379(6) Å. These values are similar to the bond lengths observed in the six outer Ni(II) units of NiClust-SG-20. However, the Cu—N bond length in Cu(CTEA) was reported to be 2.466(5) Å, which is longer than what we observed for Ni—N in NiClust-SG-20 [2.120(6) 2.167(7) Å]0.21 Bond lengths in NiClust-SG-20 were also consistent with the reported Ni—O (2.044 to 2.046 Å), Ni—N (2.050 to 2.137 Å), and Ni—S (2.394 to 2.403 Å) bond lengths of another Ni complex, Ni-1′-Ac₂ (see FIG. 9B). Ni-1′-Ac₂ shows a similar chemical environment to the NS₂O₃ units present in NiClust-SG-20.³⁷

Although the structure of NiClust-SG-20 revealed information how SG-20 can bind to Ni(II), it is unlikely that the cluster is the predominant species in solution. Rather, mononuclear Ni-SG-20 species are likely in a complex equilibrium with multinuclear complexes in solution, where NiClust-SG-20 was isolated because it was a thermodynamically stable and crystalline product. However, the electronic absorption spectra of the green crystals of NiClust-SG-20 showed the same characteristic peak at λ=260 nm in H₂O, as seen in Ni-SG-20 and in solution from our titration experiments. Therefore, we can conclude that the characteristic peak at λ=260 nm observed in Ni-SG-20, NiClust-SG-20, and in solution from our titration experiments of Ni(II) against SG-20 is likely due to Ni binding with N, S, and O atoms, which is consistent with other known Ni(II) complexes.³⁷

### 1.4 Discussion

In this section, the synthesis, characterization, and metal-binding studies of SG-20, a highly selective chelator of Ni(II) in aqueous solution is reported. SG-20 shows high selectivity and affinity toward Ni(II) compared to other metal ions. Interestingly, SG-20 showed a stronger affinity toward Ni(II) than other structurally similar ligands like NS-1 and NiCast. One reason for the greater affinity of SG-20 may be the presence of a more basic aliphatic N atom, which is a stronger electron donor than the aniline-type N donors present in NS-1 and NiCast. Moreover, SG-20 contains an additional S and O arm compared to NS-1 and NiCast, which provides SG-20 with more donor atoms for binding Ni(II). It is clear from the structure of NiClust-SG-20 that only two of the S and O arms bind to Ni(II), forming a NS₂O₂ donor set where the octahedral field for Ni(II) is completed with an axial aqua ligand, rather than a S or O donor atom from the third arm of SG-20. However, the extra binding sites available on SG-20 likely lead to multiple interactions between SG-20 and Ni(II), which synergize and increase the overall affinity of SG-20 toward Ni(II) through the avidity effect, where the third donor arm increases the local concentration of donor atoms for Ni(II).⁵⁰ Although we were not successful in crystallizing the mononuclear complex formed between Ni(II) and SG-20, electronic absorption spectra, Job analysis, ESI mass spectra are consistent with a mononuclear Ni-SG-20 species forming in solution. However, attempts to crystallize this mononuclear complex were not successful, which may indicate that a mononuclear SG-20 Ni(II) complex is in equilibrium with higher-order species in solution. Interestingly, we were able to obtain single crystals suitable for X-ray crystallographic analysis by slow evaporation of the solvent from a 1:1 solution of Ni(II) and SG-20. These data revealed formation of NiClust-SG-20, a Ni cluster formed between SG-20 and Ni(II) that contains 15 Ni centers bound to 6 SG-20 molecules.

The salient feature of NiClust-SG-20 was that it contained two distinct types of Ni in its outer and inner core. The outer core comprised six Ni centers each of which was bonded to two of the SO arms of SG-20. The third SO arm does not bind to the same Ni center but instead binds to other Ni centers via carboxylate bridges (FIG. 9C), making up the inner core of NiClust-SG-20 with nine Ni centers. The inner core also comprised oxo bridges and two carbonate groups. Another structurally similar ligand to SG-20, NTP forms a Ni cluster with four Ni centers, [Ni₄(μ₃—OH)₂(H₂O)₆(NTP)₂] with Ni(II) (FIG. 9A). The difference in the structure of NiClust-SG-20 compared to [Ni₄ (μ3-OH)₂(H₂O)₆(NTP)₂] can be attributed to more flexibility present in the third SO arm of SG-20, as compared to NTP. Moreover, SG-20 contains three Na(I) atoms that also take part in the cluster formation in NiClust-SG-20 (FIG. 8). Therefore, this unique cluster forming ability of SG-20 may provide insights into its affinity toward Ni(II). This property also makes SG-20 an excellent candidate to be conjugated to suitable supports to selectively remove Ni(II) from extracts of fly ash.

### 1.5 Conclusions

In conclusion, we report the synthesis of SG-20, a highly selective chelator for Ni(II). SG-20 binds selectively with Ni(II) compared to other metal ions like Cu(II), Fe(II), Zn(II), and Co(II). Studies on the pH dependence of the chelating ability of SG-20 reveal that SG-20 binds tightly to Ni(II) at higher pH values (pH ˜7), with a single-digit micromolar K_{d} value. However, upon reducing the pH, the binding ability decreases drastically, which may aid in Ni(II) release. Electronic absorption spectra, Job plot analysis, and ESI mass spectra are all consistent with SG-20, forming a mononuclear Ni(II) complex in solution. Even though single crystals of a mononuclear complex were not obtained, we were successful in obtaining single crystals of a cluster formed between Ni(II), Na(I), and SG-20. It was evident from the structure of NiClust-SG-20 that two of the NSO arms of SG-20 are primarily involved in binding with Ni(II). The third NSO arm does not participate in binding with the same Ni atom as the other two arms but binds to other Ni atoms by building oxo bridges. This unique ability of SG-20 to bind tightly and selectively to Ni(II), while carrying a third arm to bridge to larger Ni clusters, makes SG-20 an excellent candidate for capturing Ni(II) in solution. Current research in our laboratories is examining the conjugation of SG-20 to suitable carriers to selectively remove Ni(II) from solution.

### 2. Semi-Synthetic Proteins as Metal Ion Capture Agents: Catch and Release of Ni(II) and Cu(II) with Myoglobin Bioconjugates

### 2.1 Introduction

In this section, attaching metal ion chelators are attached to proteins in a semi-synthetic fashion could raise the overall efficiency of metal harvest by increasing the number of metal ions captured per protein molecule. Myoglobin (Mb) was chosen as a model protein to evaluate the semi-synthetic protein/metal ion capture strategy. Mb is a relatively small protein (17 kD) that has 10 lysine (Lys) residues on its surface, making it an attractive candidate for bioconjugation.⁵¹⁻⁵³ Additionally, recent work proved that Mb can be abundantly expressed in Nicotiana benthamiana leaves and isolated via low-cost methods, making Mb an attractive, renewable, and green protein starting material for manufacturing metal-ion capture agents.⁵⁴ In general, plants are inexpensive, green, and renewable sources of protein expression on an industrial scale due to their ability to support efficient synthesis of proteins from eukaryotic sources with scalability, relatively low production costs, and small environmental footprints.^{55,56}

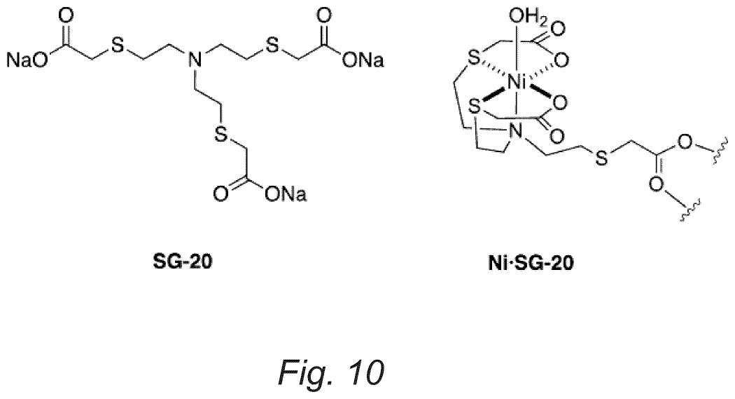

In this section, the synthesis and characterization of a semi-synthetic protein designed for Ni(II) and Cu(II) capture is also provided. The protein was obtained by conjugation of Mb to our previously synthesized compound SG-20 (FIG. 10), a chelator with high affinity for Ni(II) and Cu(II).⁵⁷ The protein conjugate Mb-SG-20 contains multiple equivalents of SG-20 covalently linked to its surface. Binding and recovery properties of Ni(II) and Cu(II) for the modified protein Mb-SG-20 were characterized, which established Mb-SG-20 as a recyclable agent. Interestingly, the affinity of Mb-SG-20 for Ni(II) was roughly the same as that of SG-20, indicating that conjugation does not compromise the affinity of the chelator toward Ni(II). However, Mb-SG-20 showed a significant increase of affinity toward Cu(II) compared to SG-20. Finally, a proof-of-concept experiment with CFA leachates showed that Mb-SG-20 could deplete Ni(II) and Cu(II) selectively from a complex solution of metal ions.

### 2.1 Experimental Section

2.1.1 Materials. Mb from equine septal muscle was purchased from Sigma-Aldrich. SG-20 was synthesized using methods previously developed in our lab.⁵⁷ Amicon Ultra-0.5 centrifugal filter units [10 kDa molecular weight cutoff (MWCO)] were purchased from Sigma-Aldrich. Nitric acid (70%, >99.9% trace metal basis) was procured from Sigma-Aldrich. NiCl₂·6H₂O was purchased from Fischer Scientific, whereas CuCl₂·2H₂O was purchased from Sigma-Aldrich. 10 ppm Ni and Cu standards (in 2 and 3% HNO3, respectively) were purchased from Inorganic Ventures.

2.1.2 Instrumentation. Inductive coupled plasma mass spectrometry (ICP-MS) analysis was performed using an Agilent 7700×ICP-MS. Synthesis of Mb-SG20. Mb (20 mg) was added to phosphate buffered saline (PBS) (10 mL, pH=7.1), containing 1-ethyl-3-(3-dimethylamino-propyl) carbodiimide (EDCI) (191 mg), ensuring the final concentration of Mb to be 2 mg/mL and that of EDCI to be 100 mM (19.1 mg/mL). To this solution was added SG-20 (100 mg) with the final concentration of SG-20 being 10 mg/mL. The resultant solution was incubated for 12 h at 37° C. The solution was purified by centrifuging through an Amicon Ultra size exclusion filter (10 kDa, MWCO) at 14,000 g for 30 min followed by washing with PBS (five times).

2.1.3 Ni(II) and Cu(II) Binding Studies of Mb. Different concentrations of NiCl₂·6H₂O, and CuCl₂·2H₂O (20-80 μM) were centrifuged either alone or in the presence of different concentrations of Mb (10 μM), through an Amicon Ultra size exclusion filter (10 kDa MWCO) at 14,000 g for 30 min. The Ni and Cu concentration (in ppb) of the resulting filtrates were measured by ICP-MS after performing a 100-fold dilution with 2% trace metal grade HNO₃.

2.1.4 Ni(II) and Cu(II) Binding Studies of Mb-SG20. Different concentrations of NiCl₂·6H₂O and CuCl₂·2H₂O (20-80 μM) were centrifuged either alone or in the presence of different concentrations of Mb-SG20 (5-40 μM), through an Amicon Ultra size exclusion filter (10 kDa MWCO) at 14,000 g for 30 min. The Ni and Cu concentrations (in ppb) of the resulting filtrates were measured by ICP-MS after performing a 100-fold dilution with 2% trace metalgrade HNO₃.

2.1.5 Mb-SG20 Recyclability Studies. NiCl₂·6H₂O (40 μM) or CuCl₂·2H₂O (60 μM) was centrifuged either alone or in the presence of Mb-SG20 (10 μM for NiCl₂·6H₂O and 20 μM for CuCl₂·2H₂O), through an Amicon Ultra size exclusion filter (10 kDa MWCO) at 14,000 g for 30 min. The Ni(II) or Cu(II) bound to Mb-SG20 remains in the residue, whereas the free Ni(II) or Cu(II) goes through the filter into the filtrate. The Ni or Cu concentration of the filtrate was analyzed by ICP-MS analysis after performing a 100-fold dilution with 2% trace metal basis HNO3. The residue was treated with ethylenediaminetetraacetic acid (EDTA, 10 mM, pH=7.1) and allowed to incubate at ambient temperature for 24 h to remove Ni(II) or Cu(II), followed by centrifugation through an Amicon Ultra size exclusion filter (10 kDa MWCO) at 14,000 g for 30 min. The Ni or Cu concentration of the filtrate was then measured by ICP-MS, after performing a 100-fold dilution with 2% trace metal basis HNO3, whereas the residue was again treated with Ni(II) or Cu(II), ensuring the Ni(II) concentration to be 40 μM or that for Cu(II) to be 60 μM. The final concentration of Mb-SG20 was fixed at 10 or 20 μM for Ni(II) and Cu(II), respectively. The aforementioned cycle was repeated twice.

2.1.6 Preparation of Coal Fly Ash Leachate Solution. The CFA used in this study was produced in a coal-fired power plant in California, USA. The material was purchased from a commercial vendor (Diversified Minerals, Inc.; Oxnard, CA) and was classified as class F (<7% CaO) based on ASTM C618 testing. Fly ash was used as received and leached for 24 h with an orbital shaker using 4 M (GR ACS) hydrochloric acid with a liquid-to-solid ratio of 6:1 mL/g. The resultant slurry was filtered using vacuum filtration with a porcelain Buchner funnel and Whatman 41 filter paper. The filtrate was filtered a second time through 0.22 μm syringe filters (25 mm polypropylene, Thermo Scientific). This solution (leachate) was stored in a sealed glass container until use in experiments. The solution was used within 2 weeks of preparation, and no precipitates were observed in the fly ash leachate.

2.1.7 Studies on Selective Ni(II) and Cu(II) Capture by Mb-SG20 from CFA Leachate Solution. To a yellow CFA leachate solution (6 mL), NaOH beads were added to raise the pH of the solution to 5.5. A brown-colored precipitate formed. The solution was centrifuged at 3000 rpm for 20 min. The colorless supernatant was collected, and the brown-colored residue was discarded. The supernatant was centrifuged through an Amicon Ultra size exclusion filter (10 kDa MWCO) at 14,000 g for 30 min, either alone or in the presence of Mb-SG20 (5-20 μM). The concentration of different types of metal ions, including lanthanides, in the filtrate, was determined by ICP-MS after performing a 50-fold dilution with 2% trace metal basis HNO₃.

2.1.8 Proteomic Analysis. (A) Protein digestion: For tandem mass spectrometry 16 μg of SG20 coupled protein was diluted in 10 μL triethylammonium bicarbonate (TEAB) (40 mM) buffer containing 5 mM dithiothreitol (DTT) and 0.01% ProteaseMax. Samples were incubated with DTT for 30 min at 65° C. followed by alkylation with iodoacetamide (15 mM) for 30 min at ambient temperature in the dark. Proteins were digested with trypsin (0.4 μg) overnight at 37° C. Following digestion, the samples were acidified by the addition of 0.5% trifluoroacetic acid (TFA) and submitted for mass spectrometry. (B) LC-MS/MS: Peptides in the digests were diluted in 0.1% formic acid/0.005% trifluoroacetic acid and desalted/separated using reverse phase chromatography (Acclaim PepMap RSLC column, Thermo) under acidic conditions (0.1% formic acid) with an EASY nLC-1000 uHPLC system (Thermo). Peptides were separated over a 45 min gradient and analyzed with an OrbiTrap QExactive mass spectrometer (Thermo). MS1 scans were performed in profile mode at 70,000 resolution. Data-dependent MS2 acquisitions were triggered on the top 10 most abundant ions (charge states 2-7) and fragmented using HCD at 30% collision energy with spectra acquired at 17,500 resolutions. Dynamic exclusion was set at 40 s.

2.1.9 Protein Identification and Quantification. LC-MS/MS data were analyzed using Proteome Discoverer (Thermo, version 2.4). Peptide identifications were scored using Sequest HT against a reviewed human protein database (Uniprot; downloaded on 2021 Mar. 30; 20,310 entries) and simultaneously against a matched decoy database to determine the false discovery rate (FDR). Searches included up to two missed tryptic cleavages and 10 ppm/0.05 Da mass tolerances for parent and fragment ions, respectively. The iodoacetamide derivative of cysteine was specified as a fixed modification. Oxidation of methionine was specified as a variable modification. SG20 modification of lysine in apo form (352.0347 Da) or with a single Na⁺ (375.0245 Da) or K⁺ (390.9984 Da) ion were specified as variable modifications. Peptides were considered a positive identification if they achieved a ≤0.1 FDR using the Percolator algorithm.

### 2.2 Results and Discussion

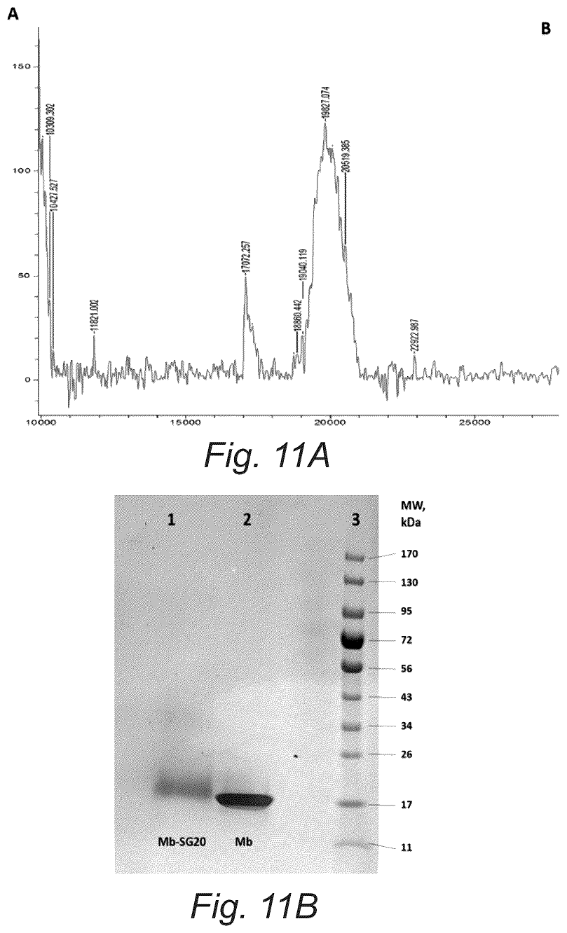

2.2.1 Synthesis, Molecular Weight Determination, and Proteomic Analysis of Mb-SG-20. The protein conjugate Mb-SG-20 was synthesized using Mb (2 mg/mL), SG-20 (10 mg/mL), and 1-ethyl-3-(3-dimethylamino-propyl)-carbodiimide (EDCI, 100 mM, 19.1 mg/mL) in PBS (pH=7.1). After incubation for 12 h at 37° C., the protein was isolated via size exclusion filtration. The covalent modification of Mb with SG-20 was confirmed using matrix-assisted laser desorption/ionization (MALDI) analysis, which revealed peaks at m/z: 18,860, 19,040, 19,827, and 20,519. These data are consistent with a distribution of 4-7 equiv of SG-20 conjugated per molecule of Mb (m/z: 17,027) (FIG. 11A). The semi-synthetic protein conjugate Mb-SG-20 was further characterized by sodium dodecyl sulfate polyacrylamide gel electrophoresis (SDS-PAGE) analysis (FIG. 11B). Unlike the purified protein Mb, which shows up as a sharp band (lane 2), Mb-SG-20 appears as broader smear (lane 3), indicating that a distribution of protein species with molecular weights greater than Mb alone are present. Importantly, the absence of intense larger molecular weight bands above ˜30 kDa in lane 3 indicated that little to no oligomerization of Mb occurred, which could arise from intermolecular amide bond coupling between SG-20 and lysine residues from two or more equivalents of Mb.

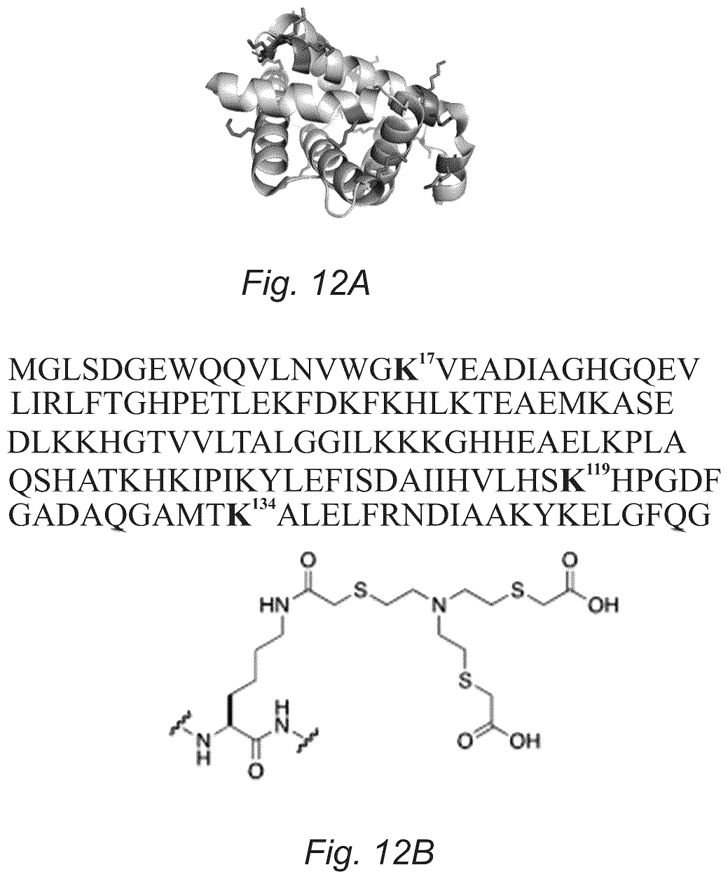

To gain further insight into how Mb was modified by SG-20, proteomic analysis was performed. Trypsin digestion identified three sites Lys17, Lys119, and Lys134 that showed SG-20 conjugated to the surface Lys residues through the formation of an amide bond (FIG. 12A,B). Unfortunately, chymotrypsin digestion did not produce good coverage, and no other sites conjugated to SG-20 were identified from this digest. The inability to identify other sites of covalent modification may be due to intramolecular cross-linking, where one unit of SG-20 formed amide bonds between two adjacent Lys units on Mb, which prevented efficient digestion.

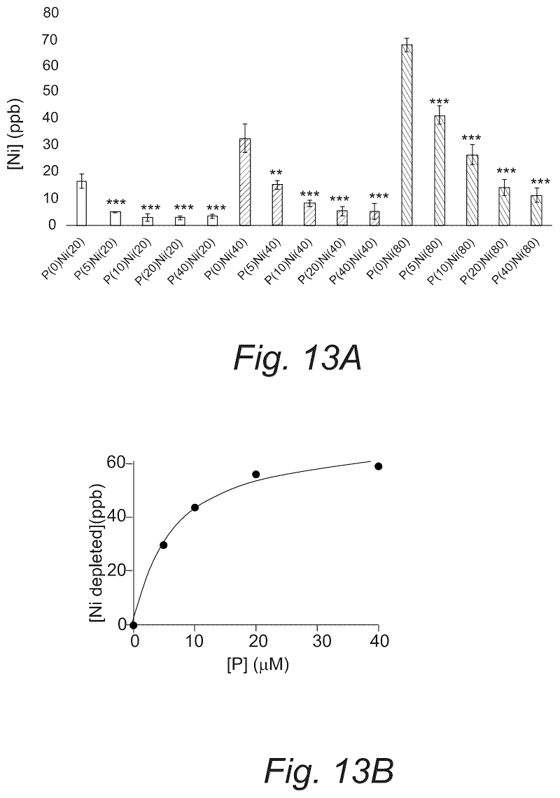

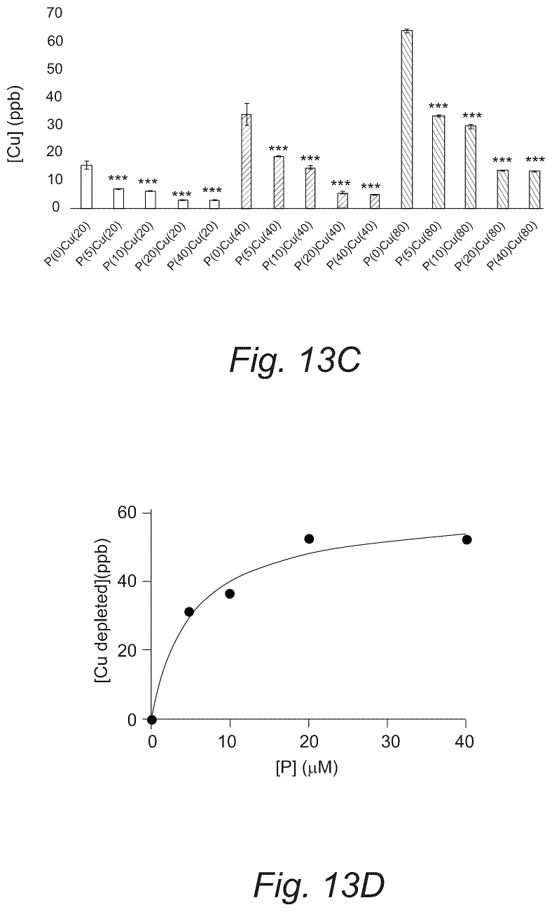

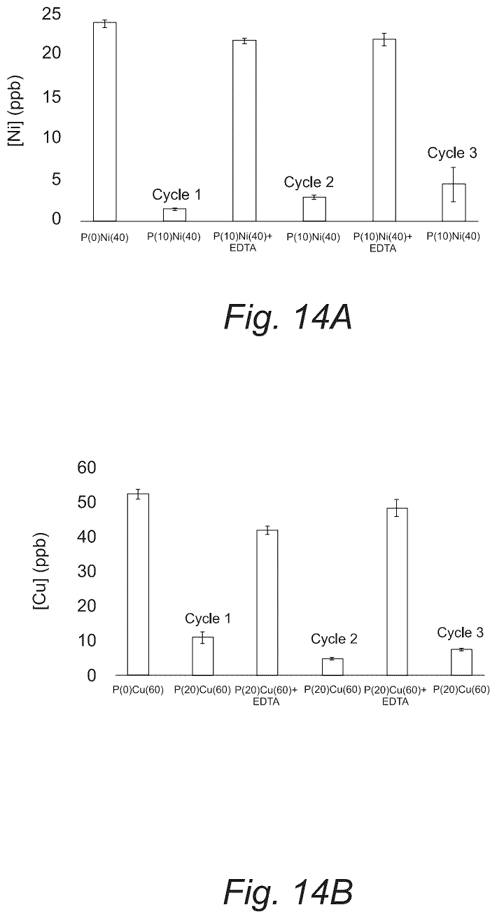

2.2.2 Binding Studies of Mb-SG-20 with Ni(II) and Cu(II). The ability of Mb-SG-20 to harvest Ni(II) and Cu(II) was evaluated. No Ni(II) or Cu(II) binding was observed with Mb alone (10 μM). Ni(II) and Cu(II) binding studies of Mb-SG-20 were performed using Ni(II) or Cu(II) (20-80 μM) and Mb-SG-20 (5-40 μM) in PBS (pH=7.1). After treatment of Ni(II) or Cu(II) solutions with Mb-SG-20 or PBS as a control, the protein was isolated by size-exclusion filtration (10 kDa cutoff filter), and Ni(II) or Cu(II) concentrations of filtrates were determined (in ppb) after 100-fold dilution by ICP-MS. ICP-MS data show that Mb-SG-20 (designated as P in FIGS. 13 and 14) was effective in removing super-stoichiometric amounts of Ni(II) and Cu(II) from solution. Importantly, Ni(II) harvest proceeded in a concentration-dependent manner. At a concentration of 5 μM, Mb-SG20 was able to remove 55-70% of Ni(II) from a solution containing 20-80 μM Ni(II) (FIG. 13A). Greater amounts of Ni(II) ion capture were achieved using greater concentrations of Mb-SG-20 (10-40 μM) and Ni(II) (20-80 μM). Moreover, when the amount of Ni(II) depleted (in ppb) from solution of fixed Ni(II) concentration (80 μM) was plotted vs the concentration of Mb-SG-20 and the data were fit to the Hill equation, an apparent Kd value of 6.3±0.8 μM was obtained (FIG. 13B), which was in good agreement with the data for SG-20 (Kd=7.0±0.2 μM).26 These results indicated that the affinity of SG-20 and Mb-SG-20 toward Ni(II) were about the same, establishing that Mb-SG-20 has high affinity for Ni(II). Cu(II) removal from solution by Mb-SG-20 was also concentration dependent. About 55% of Cu(II) (20 μM) was removed from solution using 5 μM Mb-SG-20. Interestingly, increasing the concentration of Mb-SG-20 to 10 μM did not lead to significant Cu(II) depletion. However, further increasing the concentration of Mb-SG-20 (20-40 μM) resulted in depletion of about 80% Cu(II) from solution, with varying concentration of Cu(II) (20-80 μM) (FIG. 13C). Plotting the concentration of Mb-SG-20 versus the concentration of Cu(II) depleted from a solution containing fixed Cu(II) concentration (80 μM), and subsequently fitting the data to the Hill equation, resulted in an apparent K_{d} value of 5.2±0.5 μM (FIG. 13D).

2.2.3 Studies on the Recyclability of Mb-SG-20. We tested the hypothesis that Mb-SG-20 could act as a recyclable metal harvesting reagent. A solution of Mb-SG-20 (10 μM) and Ni(II) (40 μM) was passed through a 10 kDa size exclusion filter, the filtrate was collected and the residue containing Ni(II)-bound Mb-SG-20 was treated with EDTA (10 mM, PBS, pH=7.1). The resultant solution was incubated for 24 h to strip Ni(II) from Mb-SG-20, then the protein solution was concentrated via size-exclusion filtration, and the filtrates were analyzed by ICP-MS. Greater than 70% of the Ni(II) from a 40 μM solution is captured after treatment with 10 μM Mb-SG-20 (P (10) Ni (40), FIG. 14A); upon treatment with EDTA that Ni(II) is harvested in the filtrate (P (10) Ni (40)+EDTA, FIG. 14A). To test for recyclability, the Ni(II) and EDTA treatment was repeated for a second and third cycle, and similar results were obtained (see Experimental Section for detailed experimental procedures). Similar results were obtained upon performing the same set of experiments with Mb-SG-20 (20 μM) and Cu(II) (60 μM) (FIG. 14B). Greater concentrations of Mb-SG-20 were chosen because of the concentration-dependent binding of Mb-SG-20 with Cu(II), as seen from our binding studies data that reveal that higher concentrations of Mb-SG-20 are required for maximum depletion of Cu(II) compared to Ni(II). Because we chose a large concentration of Mb-SG-20 for recyclability studies with Cu(II), we increased the concentration of Cu(II) for these experiments. Overall these results indicate that Mb-SG-20 captures both Ni(II) and Cu(II), then releases them upon treatment with EDTA, then binds and releases them again through at least three cycles to act as a recyclable agent.

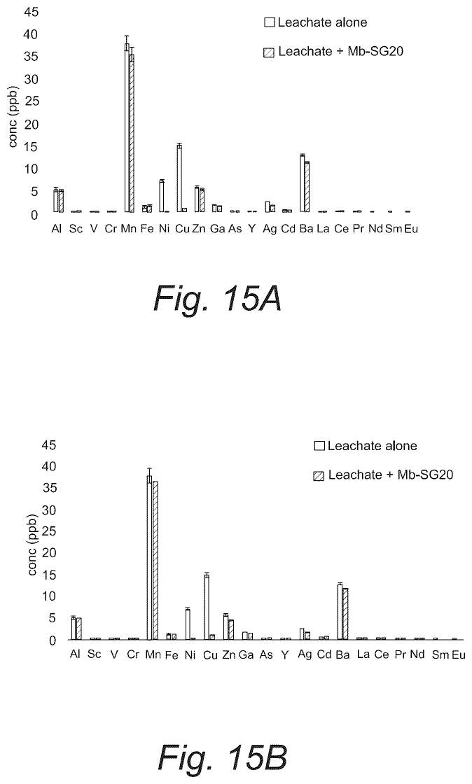

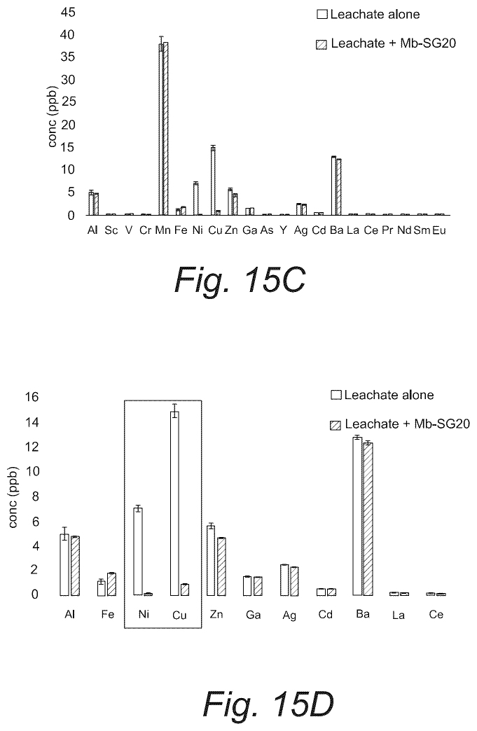

2.2.4 Selective Extraction of Ni(II) and Cu(II) from CFA. Having established that Mb-SG-20 is an effective and recyclable agent for removing Ni(II) from solution, we tested the ability of Mb-SG-20 to capture Ni(II) and Cu(II) selectively from a complex mixture of metal ions. CFA leachates, which contain valuable lanthanides and transition metal ions, were chosen for this proof-of-concept experiment. A CFA leachate solution was prepared by digestion of CFA with HCl (4 M). The pH of the CFA leachate was raised to pH=5.5 using solid NaOH beads to ensure minimum dilution of the solution. The pH value of 5.5 was chosen because it was shown previously with SG-20 to be within the optimal range for Ni(II) binding.²⁶ The leachate was centrifuged to remove solids, and the supernatant was treated with Mb-SG-20 (5-20 μM). Size-exclusion filtration (10 kDa cutoff) of the leachate alone (control) and leachate treated with Mb-SG-20 were performed. Metal ion concentrations of the filtrates were analyzed by ICP-MS. ICP-MS analysis revealed that almost all the Ni and Cu present in the leachate at pH=5.5 was removed by Mb-SG-20. Interestingly, none of the other metal ions competed with Ni and Cu, even at higher concentrations of Mb-SG-20 (FIG. 15A-D). Moreover, our data are intriguing because the concentration of Mn in the CFA leachate exceeded that of Ni and Cu by a factor of 5.4 and 2.5, respectively. These results demonstrate that Mb-SG-20 captures Ni and Cu selectively from a complex mixture of metal ions.

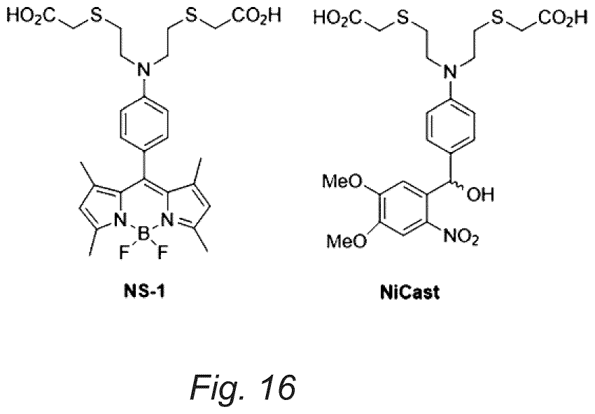

In this manuscript, we present our findings on the conjugation of the chelator SG-20, previously synthesized by us with Mb.⁵⁷ Our aim was to achieve a selective extraction of Ni(II) and Cu(II) from aqueous solutions. SG-20 was designed based on the structural characteristics of other established chelators, namely NS-1 and NiCast (FIG. 16), that exhibit affinity for Ni(II) and Cu(II) in aqueous solutions.^{58,59} Both NS-1 and NiCast contain an aromatic nitrogen with two sulfur and oxygen-containing arms. Experimentally observed Kd values for NS-1 are 193 and 0.63 μM for Ni(II) and Cu(II), respectively, whereas the values for NiCast are 560 and 13 μM, respectively, for Ni(II) and Cu(II). Another ligand structurally similar to NS-1 and NiCast, N,N-bis[2-(carboxylmethyl)thioethyl]amine (CTEA), was reported to have Kd values of 219 and 49 μM for Ni(II) and Cu(II), respectively.^{58,59} Collectively, these ligands containing an aromatic N-donor with two SO arms exhibited stronger affinity for Cu(II) compared to Ni(II), consistent with the Irving Williams series.

In contrast to previous chelators, SG-20 introduced a unique aliphatic nitrogen with three arms containing sulfur and oxygen donors. Strikingly, SG-20 exhibits unexpected behavior by displaying greater selectivity for Ni(II) (Kd=7.0 μM) over Cu(II) (Kd=200 μM), defying the predictions of the Irving Williams series. This intriguing finding may be attributed to the capability of the third arm to support the formation of Ni(II) clusters, as supported by the X-ray structure of the SG-20 complex with Ni(II).⁵⁷ It is worth noting that the formation of Mb-SG-20, achieved by conjugating SG-20 to the surface Lys residues of Mb, resulted in the conversion of one carboxylate donor into an amide. This alteration might explain why Mb-SG-20 exhibited similar affinity toward Ni(II) and Cu(II), with observed Kd values of 6.3 and 5.2 μM, respectively. Even though the selectivity between Ni(II) and Cu(II) binding was lower with Mb-SG-20 compared to SG-20, the semi-synthetic protein still maintained affinity for Ni(II). Furthermore, the ratio of Kd values for Cu(II) and Ni(II) was closer to unity for Mb-SG-20 (1.2) when compared to NS-1 (332), NiCast (43), and CTEA (4.5), which all bind Cu(II) much stronger than Ni(II). Further investigation is required to identify the precise structural factors governing the selectivity between Ni(II) and Cu(II) binding in these chelators.

Mb-SG-20 possesses notable characteristics that make it an effective and environmentally friendly agent for capturing and selectively removing multiple equivalents of Ni(II) and Cu(II) from aqueous solutions, particularly in complex mixtures like CFA leachates. The unique binding ability to both Ni(II) and Cu(II) enables the selective removal of the ions, even in the presence of other metal ions, such as Mn(II), that are present in greater concentrations. This binding affinity, however, does not hinder the practicality of using Mb-SG-20 because methods for the separation of Ni(II) and Cu(II) are well established.^{29,30} The recovery of Ni(II) and Cu(II) from Mb-SG-20 is a straightforward process because treatment with the inexpensive and readily available ligand EDTA enables the release of the ions. This method makes Mb-SG-20 an excellent candidate for metal ion capture and ensures environmental sustainability by minimizing the use of strong acids and significantly reducing the generation of hazardous wastes. The process to synthesize Mb-SG-20 is green because water was the only solvent used, and size exclusion filtration, rather than column chromatography, was the purification method. Size exclusion filtration can easily be carried out on the laboratory bench scale using centrifugal filters, whereas tangential flow filtration could be used on the industrial scale. Additionally, Mb-SG-20 exhibits recyclability, further enhancing its environmental friendliness in the selective separation and recovery of Ni(II) and Cu(II) from CFA leachates.

### 2.3 Conclusions

In summary, this section describes the development of Mb-SG-20, a unique Mb-conjugated protein with exceptional efficiency in capturing multiple equivalents of Ni(II) and Cu(II) from aqueous solutions. Mb-SG-20 exhibits excellent recyclability, enabling the capture and release of Ni(II) and Cu(II) over multiple cycles. The effectiveness was further demonstrated in capturing Ni(II) and Cu(II) from CFA leachate solutions containing complex mixtures of metal ions. This innovative green strategy presents an alternative approach to recover valuable materials such as Ni(II) and Cu(II), compared to environmentally destructive mining processes. The successful use of semi-synthetic proteins as metal ion capture agents in the extraction of valuable metals from CFA leachates opens possibilities for similar applications in diverse areas, including wastewater treatment. The use of semi-synthetic proteins holds promise for sustainable and environmentally friendly approaches in the harvesting of valuable metal ions from various sources, promoting resource recycling and minimizing environmental impact.

### 3. Mb-DTPA Capture Agents

### 3.1 Introduction

The semi-synthetic method of section 2 is extended to capture and release lanthanides.

### 3.2 Experimental

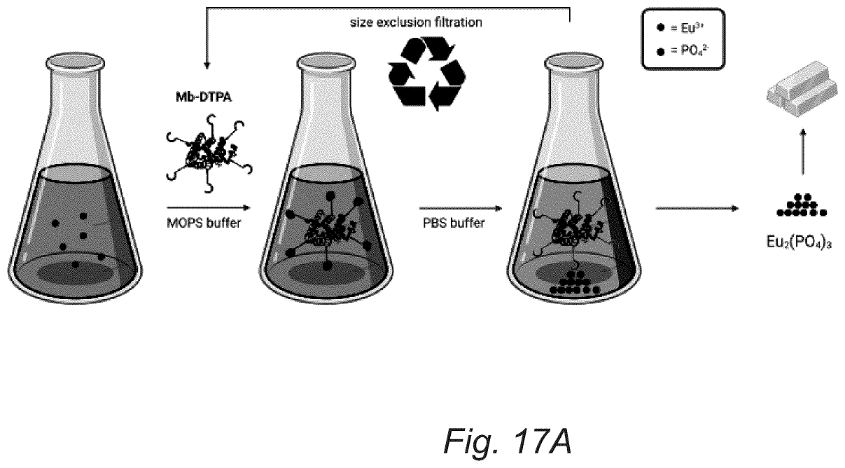

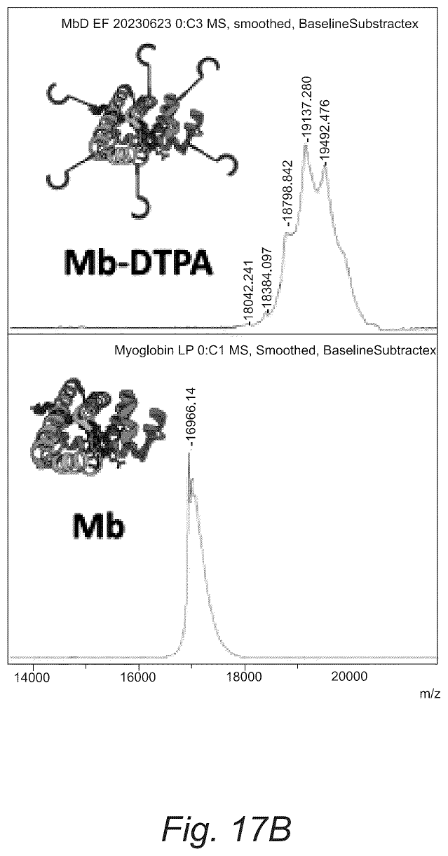

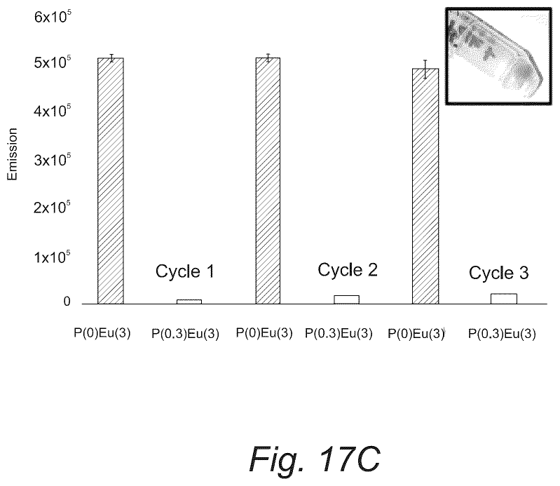

Using only Mb, DTPA anhydride, and bicarbonate buffer, we synthesized the protein conjugate Mb-DTPA. MALDI data confirmed the ligation of 5-8 equivalents of DTPA to Mb (FIG. 17B). Impressively, Mb-DTPA captured approximately 8 equivalents of Eu(III) from aqueous solutions (FIG. 17C). Unlike Mb-SG-20, which required high concentrations of EDTA to remove bound Ni(II) or Cu(II), Mb-DTPA released Eu(III) when transferred from MOPS buffer to phosphate buffer, resulting in the deposition of solid Eu₂(PO₄)₃ (see inset, FIG. 17C). The use of a soluble capture agent instead of a solid support represents a paradigm shift in metal ion capture, as it allows for facile separation of insoluble Eu₂(PO₄)₃ from the soluble protein capture agent Mb-DTPA. This method would not be practical or feasible with a chelator bound to a solid support. Importantly, our method eliminates the need for strong acids or reductants to strip the lanthanide from the chelator. To highlight the potential of green chemistry practices, a simple buffer exchange back to MOPS enables the recycling of Mb-DTPA, with little to no loss in efficiency of Eu(III) capture and release over at least three cycles (FIG. 17C). During this grant period, our objectives are to fully characterize Mb-DTPA, optimize the synthetic protocol to maximize the amount of synthetic chelator bound to the protein, establish the utility of Mb-DTPA in capturing and releasing valuable lanthanides from complex solutions, and develop innovative multi-protein separation systems to process complex samples from nontraditional sources. The overall goal is to separate transition metals from lanthanides and further advance our understanding of metal ion capture and release. While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

(1) Zaheer, M.; Schmalz, T.; Motz, G.; Kempe, R. Polymer derived non-oxide ceramics modified with late transition metals. Chem. Soc. Rev. 2012, 41, 5102-5116.
(2) Shao, Z.; Zhang, H. Combining transition metal catalysis and organocatalysis: a broad new concept for catalysis. Chem. Soc. Rev. 2009, 38, 2745-2755.
(3) Negishi, E. i. Magical power of transition metals: past, present, and future (Nobel Lecture). Angew. Chem., Int. Ed. 2011, 50, 6738-6764.
(4) Du, Z.; Shao, Z. Combining transition metal catalysis and organocatalysis—an update. Chem. Soc. Rev. 2013, 42, 1337-1378.
(5) Wang, Q. H.; Kalantar-Zadeh, K.; Kis, A.; Coleman, J. N.; Strano, M. S. Electronics and optoelectronics of two-dimensional transition metal dichalcogenides. Nat. Nanotechnol. 2012, 7, 699-712.
(6) Meyer, J.; Hamwi, S.; Kröger, M.; Kowalsky, W.; Riedl, T.; Kahn, A. Transition metal oxides for organic electronics: energetics, device physics and applications. Adv. Mater. 2012, 24, 5408-5427.
(7) Xu, X.; Liu, W.; Kim, Y.; Cho, J. Nanostructured transition metal sulfides for lithium ion batteries: progress and challenges. Nano Today 2014, 9, 604-630.
(8) Dudka, S.; Adriano, D. C. Environmental impacts of metal ore mining and processing: a review. J. Environ. Qual. 1997, 26, 590-602.
(9) Edwards, D. P.; Sloan, S.; Weng, L.; Dirks, P.; Sayer, J.; Laurance, W. F. Mining and the African environment. Conserv. Lett. 2014, 7, 302-311.
(10) Thompson, R. L.; Bank, T.; Montross, S.; Roth, E.; Howard, B.; Verba, C.; Granite, E. Analysis of rare earth elements in coal fly ash using laser ablation inductively coupled plasma mass spectrometry and scanning electron microscopy. Spectrochim. Acta, Part B 2018, 143, 1-11.
(11) Yakaboylu, G. A.; Baker, D.; Wayda, B.; Sabolsky, K.; Zondlo, J. W.; Shekhawat, D.; Wildfire, C.; Sabolsky, E. M. Microwave-assisted pretreatment of coal fly ash for enrichment and enhanced extraction of rare-earth elements. Energy Fuels 2019, 33, 12083-12095.
(12) Chaudhary, S.; Banerjee, D. Speciation of some heavy metals in coal fly ash. Chem. Speciation Bioavailability 2007, 19, 95-102.
(13) Park, D.; Middleton, A.; Smith, R.; Deblonde, G.; Laudal, D.; Theaker, N.; Hsu-Kim, H.; Jiao, Y. A biosorption-based approach for selective extraction of rare earth elements from coal byproducts. Sep. Purif. Technol. 2020, 241, 116726.
(14) Dong, Z.; Mattocks, J. A.; Deblonde, G. J.; Hu, D.; Jiao, Y.; Cotruvo, J. A., Jr.; Park, D. M. Bridging Hydrometallurgy and Biochemistry: A Protein-Based Process for Recovery and Separation of Rare Earth Elements. ACS Cent. Sci. 2021, 7, 1798-1808.
(15) Hovey, J. L.; Dardona, M.; Allen, M. J.; Dittrich, T. M. Sorption of rare-earth elements onto a ligand-associated media for pH dependent extraction and recovery of critical materials. Sep. Purif. Technol. 2021, 258, 118061.
(16) Gupta, C. K.; Krishnamurthy, N. Extractive metallurgy of rare earths. Int. Mater. Rev. 1992, 37, 197-248.
(17) Levason, W.; McAuliffe, C. Bidentate ligands containing very soft donor atoms. Nickel (II) complexes of arylarsines and arylstibines. Inorg. Chim. Acta 1974, 11, 33-40.
(18) Liu, X.; Lin, Q.; Wei, T.-B.; Zhang, Y.-M. A highly selective colorimetric chemosensor for detection of nickel ions in aqueous solution. New J. Chem. 2014, 38, 1418-1423.
(19) Kang, J. H.; Lee, S. Y.; Ahn, H. M.; Kim, C. A novel colorimetric chemosensor for the sequential detection of Ni2+ and CN− in aqueous solution. Sens. Actuators, B 2017, 242, 25-34.
(20) Dodani, S. C.; He, Q.; Chang, C. J. A turn-on fluorescent sensor for detecting nickel in living cells. J. Am. Chem. Soc. 2009, 131, 18020-18021.
(21) Hickey, E. E.; Kennedy, D. P.; Gwizdala, C.; Basa, P. N.; Müller, P.; MacDonald, J.; Burdette, S. C. Probing the Ni2+-selective Response of Fluorescent Probe NiSensor-1 with the NiCast Photocaged Complex. Photochem. Photobiol. 2022, 98, 362-370.
(22) Kraithong, S.; Chailek, N.; Sirirak, J.; Suwatpipat, K.; Wanichacheva, N.; Swanglap, P. Improving sensitivity of a new Hg2+-Selective fluorescent sensor by silver nanoparticles via plasmonic enhancement. J. Photochem. Photobiol., A 2021, 407, 113064.
(23) Ichikawa, S.; Ueno, H.; Sunadome, T.; Sato, K.; Matsuda, A. Tris (azidoethyl) amine hydrochloride; a versatile reagent for synthesis of functionalized dumbbell oligodeoxynucleotides. Org. Lett. 2013, 15, 694-697.
(24) King, P.; Clérac, R.; Wernsdorfer, W.; Anson, C. E.; Powell, A. K. Synthesis and magnetism of oxygen-bridged tetranuclear defect dicubane Co(II) and Ni(II) clusters. Dalton Trans. 2004, 2670-2676.
(25) Sheldrick, G. M. SHELXT-Integrated space-group and crystal structure determination. Acta Crystallogr., Sect. A: Found. Adv. 2015, 71, 3-8.
(26) Sheldrick, G. M. Crystal structure refinement with SHELXL. Acta Crystallogr., Sect. C: Struct. Chem. 2015, 71, 3-8.
(27) Dolomanov, O. V.; Bourhis, L. J.; Gildea, R. J.; Howard, J. A.; Puschmann, H. OLEX2: a complete structure solution, refinement and analysis program. J. Appl. Crystallogr. 2009, 42, 339-341.
(28) King, P.; Clérac, R.; Wernsdorfer, W.; Anson, C. E.; Powell, A. K. Synthesis and magnetism of oxygen-bridged tetranuclear defect dicubane Co(II) and Ni(II) clusters. Dalton Trans. 2004, 2670-2676.
(29) Sun, M.; Hong, C.-Y.; Pan, C.-Y. A unique aliphatic tertiary amine chromophore: fluorescence, polymer structure, and application in cell imaging. J. Am. Chem. Soc. 2012, 134, 20581-20584.
(30) Athawale, V.; Lele, V. Stability constants and thermodynamic parameters of complexes of lanthanide ions and (±)-norvaline. J. Chem. Eng. Data 1996, 41, 1015-1019.
(31) Kraft, A. The determination of the pKa of multiprotic, weak acids by analyzing potentiometric acid-base titration data with difference plots. J. Chem. Educ. 2003, 80, 554.
(32) Fernandez Martinez, M. T. G. P.; Maria, J.; Gonzalez Garcia, S.; Niclos Gutierrez, J. Approach to the calculation of the dissociation constants of aminotricarboxylic acids: determination of the pKa values of nitrilotripropionic acid. Ars Pharm. 1986, 27, 455-466.
(33) Xiao, Z.; Wedd, A. G. The challenges of determining metal-protein affinities. Nat. Prod. Rep. 2010, 27, 768-789.
(34) Li, H.; Dong, W.; Liu, Y.; Zhang, H.; Wang, G. Enhanced biosorption of nickel ions on immobilized surface-engineered yeast using nickel-binding peptides. Front. Microbiol. 2019, 10, 1254.
(35) DeSilva, T. M.; Veglia, G.; Porcelli, F.; Prantner, A. M.; Opella, S. J. Selectivity in heavy metal-binding to peptides and proteins. Biopolymers 2002, 64, 189-197.
(36) Goodman, D. C.; Farmer, P. J.; Darensbourg, M. Y.; Reibenspies, J. H. A coordination polymer of nickel (II) based on a pentadentate N, S, and O donor ligand. Inorg. Chem. 1996, 35, 4989-4994.
(37) Almaraz, E.; Denny, J. A.; Foley, W. S.; Reibenspies, J. H.; Bhuvanesh, N.; Darensbourg, M. Y. Zinc/Nickel exchange and ligand cannibalism in N 2 S 2 O 1, 2 donor ligand sets. Dalton Trans. 2009, 9496-9502.
(38) Jarmoskaite, I.; AlSadhan, I.; Vaidyanathan, P. P.; Herschlag, D. How to measure and evaluate binding affinities. Elife 2020, 9, No. e57264.
(39) Stefan, M. I.; Le Novère, N. Cooperative binding. PLOS Comput. Biol. 2013, 9, No. e1003106.
(40) Job, P., Job's method of continuous variation. Ann. Chim. 1928, 9 ( )
(41) Renny, J. S.; Tomasevich, L. L.; Tallmadge, E. H.; Collum, D. B. Method of continuous variations: applications of job plots to the study of molecular associations in organometallic chemistry. Angew. Chem., Int. Ed. 2013, 52, 11998-12013.
(42) Huang, C. Y. Determination of binding stoichiometry by the continuous variation method: the job plot. Methods Enzymol. 1982, 87, 509-525.
(43) Filipský, T.; Říha, M.; Hrdina, R.; Vávrová, K.; Mladěnka, P. Mathematical calculations of iron complex stoichiometry by direct UV-Vis spectrophotometry. Bioorg. Chem. 2013, 49, 1-8.
(44) Abd El-Rahman, M. K.; Mahmoud, A. M. A novel approach for spectrophotometric determination of succinylcholine in pharmaceutical formulation via host-guest complexation with water-soluble psulfonatocalixarene. RSC Adv. 2015, 5, 62469-62476.
(45) Strathmann, T. J.; Myneni, S. C. Speciation of aqueous Ni(II)-carboxylate and Ni(II)-fulvic acid solutions: Combined ATR-FTIR and XAFS analysis. Geochim. Cosmochim. Acta 2004, 68, 3441-3458.
(46) Papageorgiou, S. K.; Kouvelos, E. P.; Favvas, E. P.; Sapalidis, A. A.; Romanos, G. E.; Katsaros, F. K. Metal-carboxylate interactions in metal-alginate complexes studied with FTIR spectroscopy. Carbohydr. Res. 2010, 345, 469-473.
(47) Gup, R.; Kιrkan, B. Synthesis and spectroscopic studies of copper (II) and nickel (II) complexes containing hydrazonic ligands and heterocyclic coligand. Spectrochim. Acta, Part A 2005, 62, 1188-1195.
(48) Sanatkar, T. H.; Khorshidi, A.; Sohouli, E.; Janczak, J. Synthesis, crystal structure, and characterization of two Cu(II) and Ni(II) complexes of a tetradentate N₂O₂ Schiff base ligand and their application in fabrication of a hydrazine electrochemical sensor. Inorg. Chim. Acta 2020, 506, 119537.
(49) Chen, Z.; Wu, Y.; Huang, F.; Gu, D.; Gan, F. Synthesis, spectral, and thermal characterizations of Ni(II) and Cu(II) β-diketone complexes with thenoyltrifluoroacetone ligand. Spectrochim. Acta, Part A 2007, 66, 1024-1029.
(50) Vauquelin, G.; Charlton, S. J. Exploring avidity: understanding the potential gains in functional affinity and target residence time of bivalent and heterobivalent ligands. Br. J. Pharmacol. 2013, 168, 1771-1785.
(51) Åkeson, Å.; Theorell, H. On the microheterogeneity of horse myoglobin. Arch. Biochem. Biophys. 1960, 91, 319-325.
(52) Azami-Movahed, M.; Meratan, A. A.; Ghasemi, A.; Ebrahim-Habibi, A.; Nemat-Gorgani, M. Acetylation of lysine residues in apomyoglobin: Structural changes, amyloid fibrillation, and role of surface charge. Int. J. Biol. Macromol. 2018, 107, 626-634.
(53) Kalia, J.; Raines, R. T. Advances in bioconjugation. Curr. Org. Chem. 2010, 14, 138-147.
(54) Carlsson, M. L.; Kanagarajan, S.; Bülow, L.; Zhu, L.-H. Plant based production of myoglobin-a novel source of the muscle hemeprotein. Sci. Rep. 2020, 10, 920.
(55) Damaj, M. B.; Jifon, J. L.; Woodard, S. L.; Vargas-Bautista, C.; Barros, G. O. F.; Molina, J.; White, S. G.; Damaj, B. B.; Nikolov, Z. L.; Mandadi, K. K. Unprecedented enhancement of recombinant protein production in sugarcane culms using a combinatorial promoter stacking system. Sci. Rep. 2020, 10, 13713.
(56) Schillberg, S.; Raven, N.; Spiegel, H.; Rasche, S.; Buntru, M. Critical Analysis of the Commercial Potential of Plants for the Production of Recombinant Proteins. Front. Plant Sci. 2019, 10, 720.
(57) Gupta, S.; Ward, C. L.; Perera, S. S.; Gowan, C. T.; Dittrich, T. M.; Allen, M. J.; McElmurry, S. P.; Kodanko, J. J. Development of a Highly Selective Ni(II) Chelator in Aqueous Solution. Inorg. Chem. 2022, 61, 19492-19501.
(58) Dodani, S. C.; He, Q.; Chang, C. J. A turn-on fluorescent sensor for detecting nickel in living cells. J. Am. Chem. Soc. 2009, 131, 18020-18021.
(59) Hickey, E. E.; Kennedy, D. P.; Gwizdala, C.; Basa, P. N.; Müller, P.; MacDonald, J.; Burdette, S. C. Probing the Ni2+-selective Response of Fluorescent Probe NiSensor-1 with the NiCast Photocaged Complex. Photochem. Photobiol. 2022, 98, 362-370.
(60) Molinari, R.; Poerio, T.; Argurio, P. Selective separation of copper (II) and nickel (II) from aqueous media using the complexation-ultrafiltration process. Chemosphere 2008, 70, 341-348.
(61) Ogwuegbu, M.; Oforka, N. Solvent extraction separation studies of iron (III), cobalt (II), nickel (II) and copper (II) from aqueous solution with 1-phenyl-3-methyl-4-(p-nitrobenzoyl)-5-pyrazolone. Hydrometallurgy 1994, 34, 359-367.

What is claimed is:

1. A chelator comprising:
a compound having formula I or a salt thereof for removing transition metal ions and/or lanthanide metal ions from a solution: 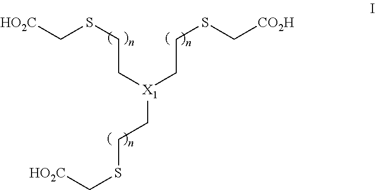
wherein:
X₁ is N or P; and
n is 1 or 2.

2. The chelator of claim 1, wherein X₁ is N.

3. The chelator of claim 1, wherein the salt is a sodium salt or a potassium salt.

4. The chelator of claim 1, wherein n is 1.

5. The chelator of claim 1 having formula II or a salt thereof:

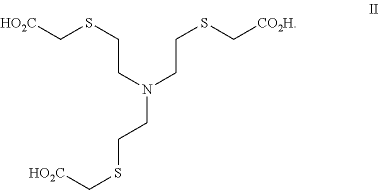 [SMILES: O=C(O)CSCCN(CCSCC(=O)O)CCSCC(=O)O]

6. A protein-chelator system for removing transition metal ions and/or lanthanide metal ions from a solution, the protein-chelator system comprising:
a chelating moiety having formula I or II or salts thereof conjugated to a protein having at least lysine residue on a surface of the protein, wherein the chelating moiety is conjugated through an amide bond formed from a nitrogen atom in the at least one lysine residue and a carboxylate moiety in the chelating moiety having formula I or III: 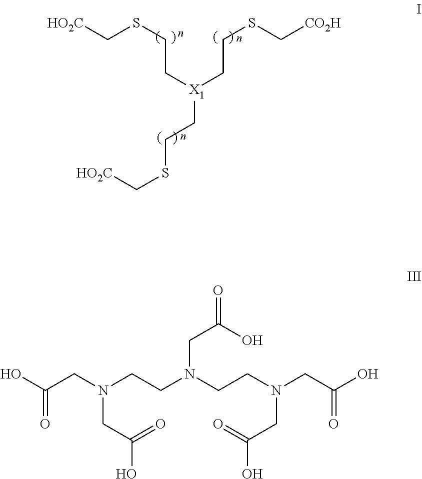
wherein:
X₁ is N or P; and
n is 1 or 2.

7. The protein-chelator system of claim 6, wherein the protein includes a plurality of lysine residues.

8. The protein-chelator system of claim 6, wherein the protein includes at least 3 lysine residues.

9. The protein-chelator system of claim 6, wherein the protein includes at least 5 lysine residues.

10. The protein-chelator system of claim 6, wherein the protein is myoglobin.

11. The protein-chelator system of claim 6, wherein the protein is an albumin.

12. A method for removing metal ions from a solution, the method comprising:
providing a target solution including transition metal ions and/or lanthanide metal ions;
contacting the target solution with a chelator having formula I or a salt thereof: 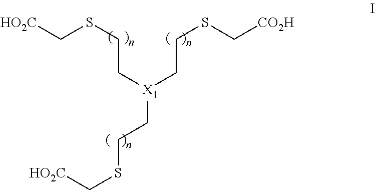
wherein:
X₁ is N or P; and
n is 1 or 2; and
collecting a precipitate that includes the transition metal ions and/or lanthanide metal ions.

13. The method of claim 12, further comprising contacting the precipitate with a release agent to recover the transition metal ions and/or lanthanide metal ions in solution.

14. The method of claim 13, wherein the release agent includes ethylenediaminetetraacetic acid.

15. The method of claim 12, wherein the transition metal ions and/or lanthanide metal ions are present in an amount of at least 0.01 μM.

16. The method of claim 12, wherein the target solution has a pH from 4 to 9.

17. The method of claim 12, wherein the transition metal ions include Ni ions and/or Cu ions and the lanthanide metal ions include europium ions.

18. The method of claim 12, wherein the target solution is an aqueous solution that includes a buffer.

19. A method for removing metal ions from a solution, the method comprising:
providing a target solution including transition metal ions or lanthanide metal ions; and
contacting the target solution with a protein-chelator system comprising:
a chelating moiety having formula I or II or salts thereof conjugated to a protein having at least lysine residue on a surface of the protein, wherein the chelating moiety is conjugated through an amide bond formed from a nitrogen atom in the at least one lysine residue and a carboxylate moiety in the chelating moiety having formula I or III:

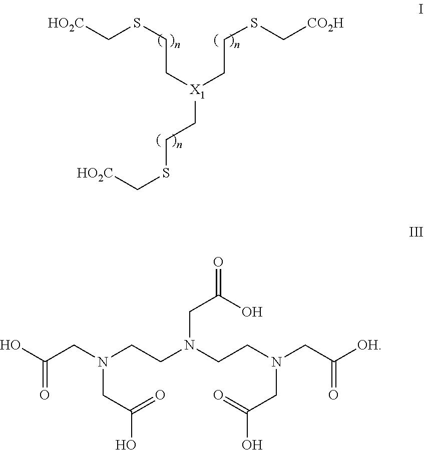

20. The method of claim 19 further comprising collecting a precipitate that includes the transition metal ions or lanthanide metal ions.

21. The method of claim 20, further comprising contacting the precipitate with a release agent to recover the transition metal ions or lanthanide metal ions in solution.

22. The method of claim 21, wherein the release agent includes ethylenediaminetetraacetic acid.

23. The method of claim 19, wherein the transition metal ions and/or lanthanide metal ions are present in an amount of at least 0.01 μM.

24. The method of claim 19, wherein the target solution has a pH from 4 to 9.

25. The method of claim 19, wherein the target solution is an aqueous solution that includes a buffer.

26. The method of claim 19, wherein the protein-chelator system is soluble in the target solution is an aqueous solution that includes a buffer.

27. The method of claim 19, wherein the transition metal ions are Ni and/or Cu.

28. The method of claim 19, wherein the protein is myoglobin.

29. The method of claim 19, wherein the protein is an albumin.

30. A method for removing metal ions from a solution, the method comprising:
providing a target solution including transition metal ions or lanthanide metal ions; and
contacting the target solution with a protein-chelator system comprising:
a chelating moiety having formula II or salts thereof conjugated to a protein having at least lysine residue on a surface of the protein, wherein the chelating moiety is conjugated through an amide bond formed from a nitrogen atom in the at least one lysine residue and a carboxylate moiety in the chelating moiety having formula III: 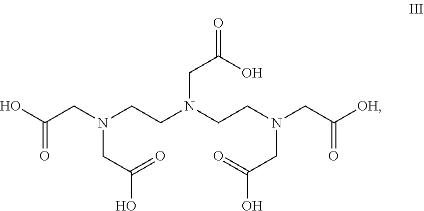 [SMILES: O=C(O)CN(CCN(CC(=O)O)CC(=O)O)CCN(CC(=O)O)CC(=O)O]
wherein the protein-chelator system is soluble in the target solution.

31. The method of claim 30, wherein the target solution is a first buffer solution.

32. The method of claim 31, wherein the first buffer solution is a HEPES buffer.

33. The method of claim 31, further comprising exchanging the first buffer solution with a second buffer solution in which chelated transition metal an/or lanthanide metal ions are insoluble.

34. The method of claim 33, wherein the second buffer solution is a phosphate buffer.

35. The method of claim 30, wherein the target solution has a pH from 4 to 9.

36. The method of claim 30, wherein the lanthanide metal ions include europium ions.

37. The method of claim 30, wherein the protein is myoglobin.

38. The method of claim 30, wherein the protein is an albumin.
